(19) United States
(12) Patent Application Publication
(10) Pub. No.: US 2026/0221280 A1
(43) Pub. Date: Jul. 30, 2026

(54) DATA-DRIVEN IMMUNE CHECKPOINT BLOCKADE THERAPY RESPONSE PREDICTION

(71) Applicant: Agency for Science, Technology and Research, Singapore (SG)

(72) Inventors: Anders SKANDERUP, Singapore (SG); Amanda Yu GUO, Singapore (SG)

(73) Assignee: Agency for Science, Technology and Research, Singapore (SG)

(21) Appl. No.: 19/143,218

(22) PCT Filed: Mar. 1, 2024
(86) PCT No.: PCT/SG2024/050120

§ 371 Date: Jun. 25, 2025

(30) Foreign Application Priority Data
Mar. 1, 2023 (SG) 102023000553X

(51) Int. Cl. G16H 50/20; G16B 25/10; G16B 40/00

(52) U.S. Cl. CPC G16H 50/20; G16B 25/10; G16B 40/00

(57) ABSTRACT

Clinical decision support systems and methods to predict clinical response of a target tumor to immune checkpoint blockade therapy (ICB), by obtaining a training dataset comprising training transcriptome records of tumor tissue samples of a plurality of known responders and a plurality of known non-responders to ICB; performing deconvolution of the training dataset to identify differentially expressed genes (DEGs) in the training dataset, wherein the DEGs are treated as features of the training dataset and associated responder, non-responder status is treated as a labels of the records in the training dataset; performing regression analysis of the features to select a subset of the features as most predictive of the responder or non-responder labels and associated feature weights; incorporating the subset of features and associated feature weights in the response estimation model; receiving transcriptome data of a target tumor tissue sample; processing the transcriptome data using the generated response estimation model to generate an estimated response metric indicating a likely response of the target tumor to ICB.

### TECHNICAL FIELD

This disclosure generally relates to methods and systems for data-driven prediction of response to immune checkpoint blockade therapy (ICB).

### BACKGROUND

This background description is provided for the purpose of generally presenting the context of the disclosure. Contents of this background section are neither expressly nor impliedly admitted as prior art against the present disclosure.

Breakthroughs in cancer immunotherapy have revolutionized cancer treatment. Immune checkpoint blockade therapy (ICB) is a promising class of immunotherapy that can induce significant tumor shrinkage and long-term disease control in some cancer patients, enabling them to maintain quality of life and continue to contribute to society.

While ICB could potentially generate a long-lasting clinical response in many previously intractable cancer types, it is an expensive therapy and not all patients benefit from ICB.

Biomarker-guided patient selection can improve the response rate and cost-benefit profile of ICB. However, the prediction accuracies of existing biomarkers are inadequate for treatment stratification in many cancer types. Furthermore, the application of existing biomarkers in the clinic are constrained by logistic and technical limitations. Consequently, it remains challenging for clinicians to identify patients who would actually benefit from ICB, highlighting an urgent need to develop new methods to aid clinician in stratifying and prioritizing cancer patients for ICB.

Known approaches for patient selection are limited and they may rely on a small set of biomarkers such as PD-L1 expression, microsatellite instability, and tumor mutation burden (TMB) of >10mutations/Mb. The known approaches are weakly predictive of treatment response and often not generalizable to cover a wider type of cancers. Clinical trials to evaluate the effectiveness of the known approaches have often yielded conflicting on inconclusive results to support effective application in clinical practice for a wider set of cancer types. Consequently, it remains challenging for clinicians to identify patients who would more likely benefit from ICB.

It would be desirable to overcome or ameliorate at least one of the above-described problems, or at least to provide a useful alternative.

### SUMMARY

Some embodiments relate to a clinical decision support system to predict clinical response of a target tumor to immune checkpoint blockade therapy (ICB), the system comprising one or more processing units to:
generate a response estimation model by:
obtaining a training dataset comprising training transcriptome data (which may comprise training transcriptome sequence records) of tumor tissue samples of a plurality of known responders and a plurality of known non-responders to ICB;
performing deconvolution of the training dataset to identify differentially expressed genes (DEGs) in the training dataset, wherein the DEGs are treated as features of the training dataset and associated responder, non-responder status is treated as a labels of the records in the training dataset;
performing regression analysis of the features to select a subset of the features as most predictive of the responder or non-responder labels and obtain associated feature weights;
incorporating the subset of features and associated feature weights in the response estimation model;
receive transcriptome sequence data of a target tumor tissue sample;
process the received transcriptome sequence data using the generated response estimation model to generate an estimated response metric indicating a likely response of the target tumor to ICB.

Some embodiments relate to a system for generating a response estimation model to predict clinical response of a tumor to immune checkpoint blockade therapy (ICB), the system comprising one or more processing units to:
obtain a training dataset comprising training transcriptome data (which may comprise training transcriptome records) of tumor tissue samples of a plurality of known responders and a plurality of known non-responders to ICB;
perform deconvolution of the training dataset to identify differentially expressed genes (DEGs) in the training dataset, wherein the DEGs are treated as features of the training dataset and associated responder, non-responder status is treated as a label of the records in the training dataset;
perform regression analysis of the features to select a subset of the features as most predictive of the responder or non-responder labels;
incorporate the subset of features in the response estimation model.

Some embodiments relate to a computer-implemented method for predicting clinical response of a tumor to immune checkpoint blockade therapy (ICB), the method comprising:
generating a response estimation model by:
obtaining a training dataset comprising training transcriptome data (which may comprise training transcriptome records) of tumor tissue samples of a plurality of known responders and a plurality of known non-responders to ICB;
performing deconvolution of the training dataset to identify differentially expressed genes (DEGs) in the training dataset, wherein the DEGs are treated as features of the training dataset and associated responder, non-responder status is treated as a label of the records in the training dataset;
performing regression analysis of the features to select a subset of the features as most predictive of the responder or non-responder labels and obtain associated feature weights;
incorporating the subset of features and associated feature weights in the response estimation model;
obtaining transcriptome data of a target tumor tissue sample;
processing the transcriptome data using the generated response estimation model to generate an estimated response metric indicating a likely response of the target tumor to ICB.

Some embodiments relate to a computer-implemented method for generating a response estimation model to predict clinical response of a tumor to immune checkpoint blockade therapy (ICB), the method comprising:
obtaining a training dataset comprising training transcriptome data (which may comprise training transcriptome records) of tumor tissue samples of a plurality of known responders and a plurality of known non-responders to ICB;
performing deconvolution of the training dataset to identify differentially expressed genes (DEGs) in the training dataset, wherein the DEGs are treated as features of the training dataset and associated responder, non-responder status is treated as a label of the records in the training dataset;
performing regression analysis of the features to select a subset of the features as most predictive of the responder or non-responder labels;
incorporating the subset of features in the response estimation model.

### BRIEF DESCRIPTION OF THE DRAWINGS

Some embodiments of systems and methods for predicting the clinical response of a target tumor to immune checkpoint blockade therapy (ICB), in accordance with the present disclosure, will now be described, by way of non-limiting example only, with reference to the accompanying drawings in which:

FIG. 1 illustrates a computer-implemented method for predicting clinical response of a tumor to ICB.

FIG. 2 illustrates a summary of data and a schematic diagram of the workflow for candidate biomarker discovery.

FIG. 3 illustrates a schematic of differentially expressed genes and pathways between responders and non-responders in the stroma. FIG. 3A illustrates a Heatmap of genes consistently differentially expressed across discovery cohorts in the stroma. Heatmaps are colored by the log 2 fold change of gene expression. P-values of individual cohorts are combined using the Fisher's method, and the −log 10 (meta p-value) is presented in the bar plot. Genes with q-value<0.01 and p-value<0.1 at least 1 ICB cohort are shown. FIG. 3B illustrates Bar plots of deconvoluted stromal expression of the top differentially expressed genes. Error bars represent >1estimated standard error. FIG. 3C illustrates Pathway enrichment of differentially expressed genes in the stroma. Bar plot shows the −log 10 (p-value) of significantly enriched pathways. Similar pathways are grouped by color. FIG. 3D illustrates Network representation of enriched pathways. Large nodes represent enriched pathways. Pathways with high similarity are fused and shown in the same color. Small nodes represent genes associated with each pathway.

FIG. 4 illustrates differentially expressed genes between responders and non-responders in the cancer compartment. FIG. 4A illustrates heatmap of genes consistently differentially expressed across discovery cohorts in the cancer cells. Heatmaps are colored by the log 2 fold change of gene expression. P-values of individual cohorts are combined using the Fisher's method, and the −log 10 (meta p-value) is presented in the bar plot. Genes with q-value<0.01 and p-value<0.1 at least 1 ICB cohort are shown. FIG. 4B illustrates bar plot shows the number of significant genes found in the stroma and cancer compartment at different q-value cutoffs. Pathway analysis of genes with cancer-specific expression differences is shown in FIG. 4C gastric cancer and FIG. 4E urothelial cancer. Bar plots of deconvoluted cancer expression of differentially expressed genes in the top enriched pathway in FIG. 4C gastric cancer and in FIG. 4E urothelial cancer. Error bars represent estimated standard error.

FIG. 5 illustrates an analysis of ligand and receptor expression of immune modulators. Heatmaps of the inferred ligand and receptor expression in the cancer and stroma compartments for immune checkpoint proteins shown in FIG. 5A and cytokines shown in FIG. 5B. Heatmaps are colored by the signed-log 10 (p-value) of differential expression.

FIG. 6 illustrates predictive modeling of response to immune checkpoint inhibition. FIG. 6A illustrates schematic of data and methods used in model training and testing. FIG. 6B illustrates top ranked features of ICB response selected by LASSO logistic regression. FIG. 6C illustrates comparisons of the performance of the IOselect model and the 3 feature model (TMB+IFNG+FCER1A) with existing biomarkers in 6 independent test cohorts. The differences between the mean AUCs of IOSelect and other models were computed using a paired t-test. ** p-value <0.01, * p-value<0.05, p-value<0.1 FIG. 6D illustrates Kaplan-Meier curves of progression free survival for patients with high vs low predicted scores (greater than or less than median); p-values from log-rank test shown.

FIG. 7 illustrates a schematic diagram summarizing the key components associated with ICB response. Red arrows indicate gene positively correlated with ICB response. Green arrow indicates gene negatively correlated with ICB response.

FIG. 8 illustrates a schematic diagram of bulk tumor expression deconvolution and permutation test to identify differentially expressed genes between responders (microsatellite instable (MSI)) and non-responders (microsatellite stable (MSS)) in both cancer and stroma compartments.

FIG. 9 illustrates identification of genes that are differentially expressed between responders and non-responders consistently across discovery cohorts. Volcano plots of differentially expressed genes as shown in the FIG. 9A for stroma and FIG. 9B for cancer compartments. The y-axis is the −log 10 (meta p-value) of differential expression. The meta p-value is calculated using the Fisher's method, combining p-values all cohorts. The x-axis is the mean log 2 (fold-change) of gene expression of responders (MSI/Epstein-Barr virus (EBV)) over non-responders (MSS). The red line represents the q-value<0.01 cutoff. Genes with median absolute log 2 (fold-change)>0.5, q-value <0.01, and p-value<0.1 in at least one ICB cohort are colored and labelled. Heatmaps of differentially expressed genes as shown in the FIG. 9C stroma and FIG. 9D cancer compartments. Heatmaps are colored by the signed-log 10 (p-value) of differentially expression. Genes with median absolute log 2 (fold-change)>0.5, q-value <0.01, and p-value<0.1 in at least one ICB cohort are shown.

FIG. 10 illustrates validation of bulk tumor expression deconvolution in ICB-treated cohorts. Inferred expression of known lineage-specific genes in cancer and stroma compartments across ICB cohorts.

FIG. 11 illustrates differential expression of the PD1 receptor and its ligands PD-L1 and PD-L2 in the stroma. Bar plots of deconvoluted stromal expression of the PD1 (PDCD1) receptor and its ligands PD-L1 (CD274) and PD-L2 (PDCD1LG2). Error bars represent estimated standard error.

FIG. 12 illustrates differentially enriched immune cell types between responders and non-responders. Boxplots showing the Cibersortx scores of 4 cell types that are significantly different between responders (MSI) and non-responders (MSS).

FIG. 13 illustrates correlations between the abundance of immune cell subtypes with the gene expression of top stromal biomarkers in individual cohorts.

FIG. 14 illustrates expression of top differentially expressed genes in immune cell subtypes. Normalized expression of 4 representative stromal biomarkers in different immune cell types. Immune cell expression data is derived from single-cell and flow cytometry sorted RNA-seq experiments from the Human Protein Atlas.

FIG. 15 illustrates a schematic diagram of IOSelect Model training and testing.

FIG. 16 illustrates benchmarking data of the IOSelect Model.

FIG. 17 a schematic of integration of the IOSelect Model with a clinical workflow.

FIG. 18 illustrates a schematic of IOSelect Model validation and refinements.

FIG. 19 illustrates a schematic diagram of the method of ICB response estimation.

FIG. 20 illustrates experimental result of tumor transcriptome deconvolution.

FIG. 21 illustrates a schematic of tumor purity estimation according to some embodiments.

FIG. 22 illustrates an evaluation of cell-type specific expression of top DEGs using single cell RNAseq data. Barplots of the percentage of cells expressing each gene for individual cell types is shown, using data from scRNAseq of renal cell cancer patients treated with ICB (Bi. et al., Cancer Cell, 2021).

FIG. 23 illustrates area under the receiver operating curve (AUC) of the cancer-DEG model on 6 test datasets. Black dot shows the mean AUC, and the error bars represent the standard error. Using one-sample t-test, we found that the predictive power of the model is not significantly different from the random expectation of AUC=0.5 (p-value=0.34).

FIG. 24 illustrates differential expression of the PD1 receptor and its ligands PD-L1 and PD-L2 in the stroma. Bar plots of deconvoluted stromal expression of the PD1 (PDCD1) receptor and its ligands PD-L1 (CD274) and PD-L2 (PDCD1LG2). Error bars represent estimated standard error.

FIG. 25 illustrates association between model prediction and overall survival. Kaplan-Meier curves of overall free survival for patients with high vs low predicted scores (greater than or less than median); p-values from log-rank test shown.

### DETAILED DESCRIPTION

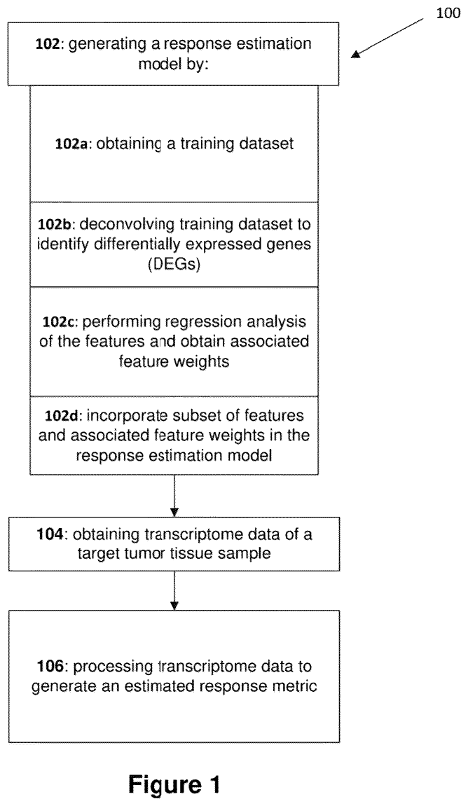

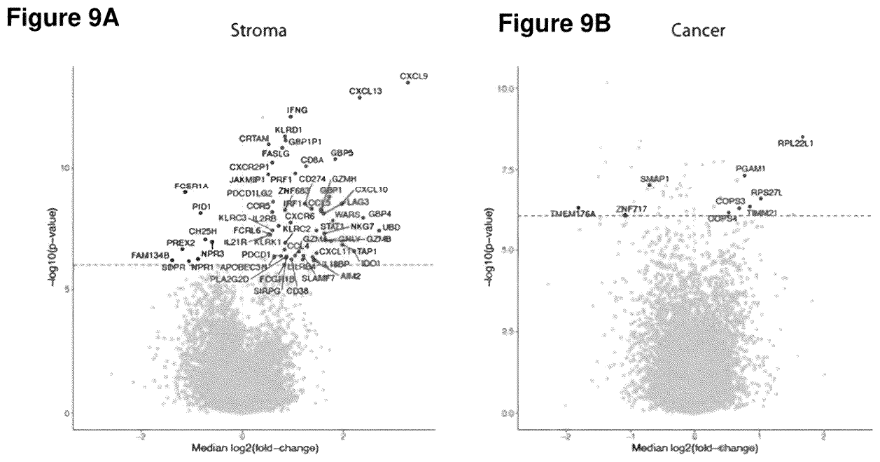

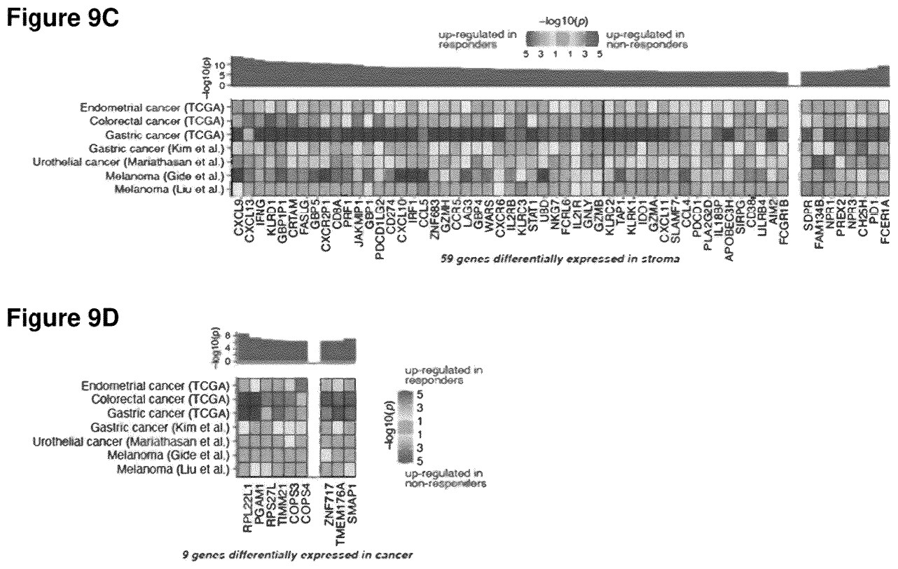

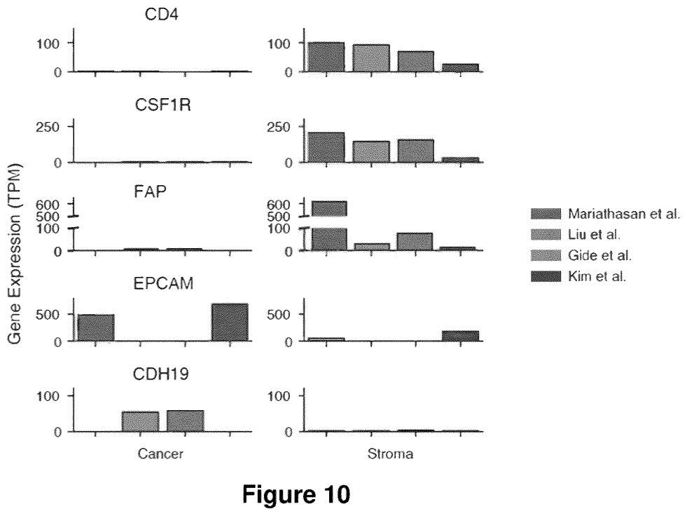

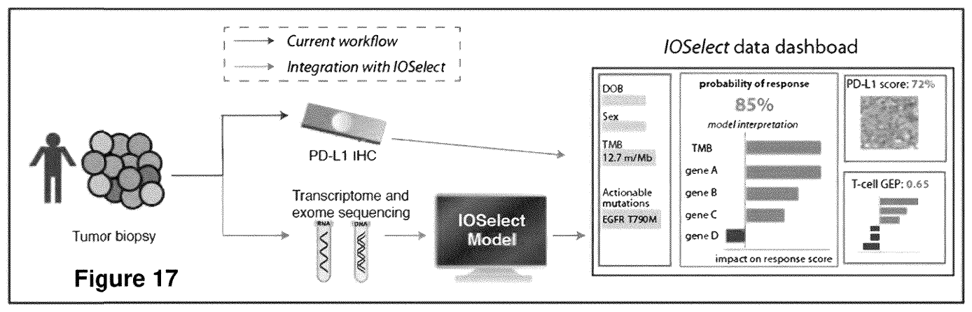

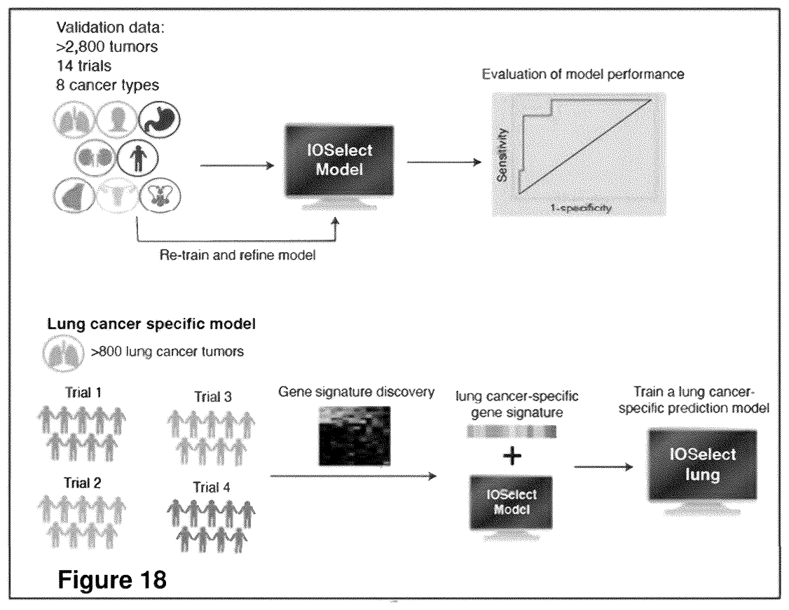

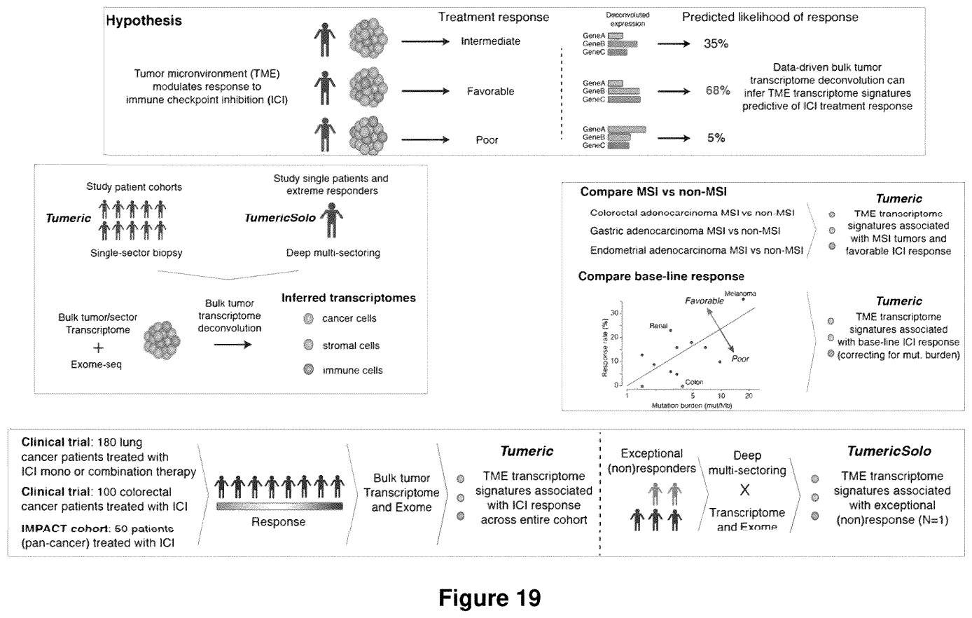

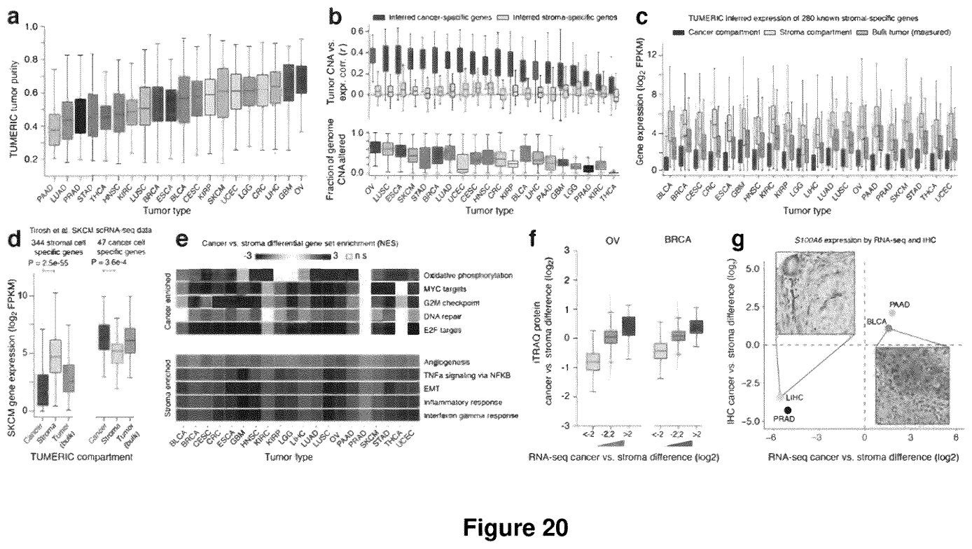

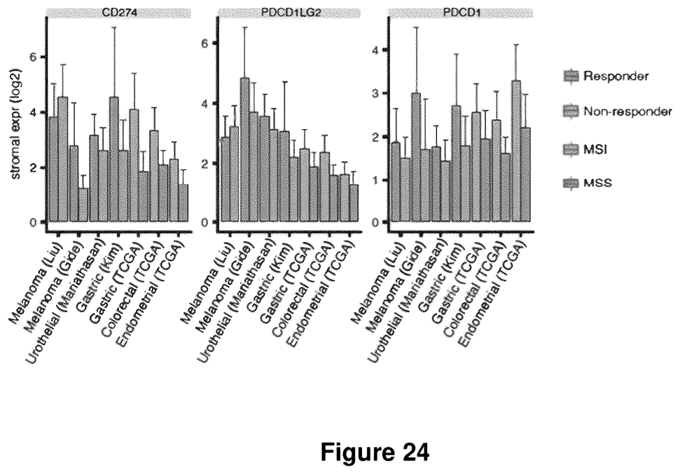

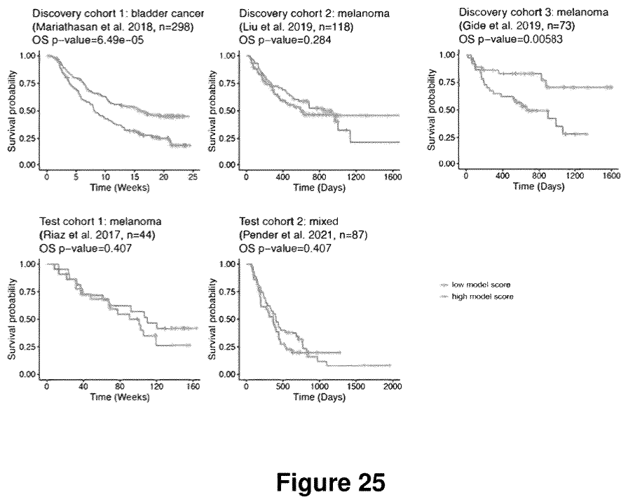

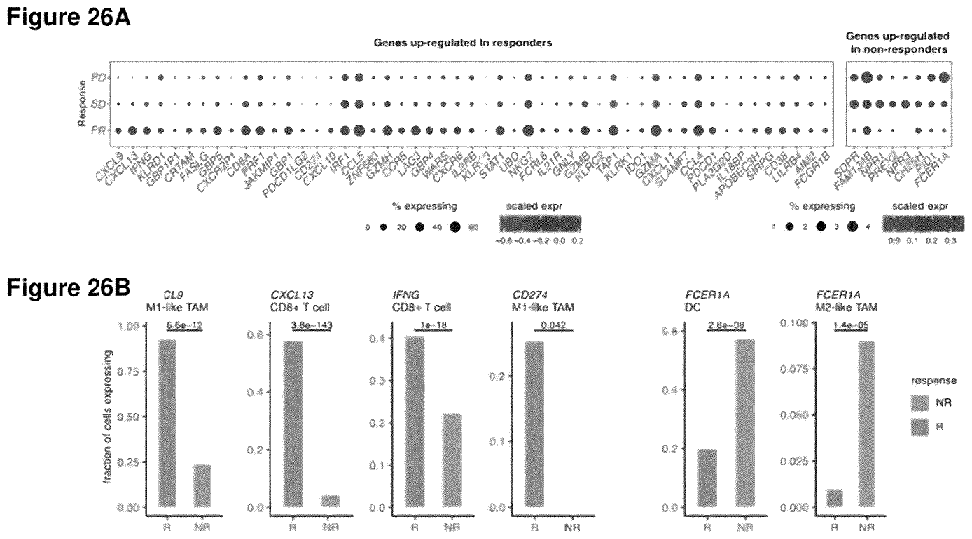

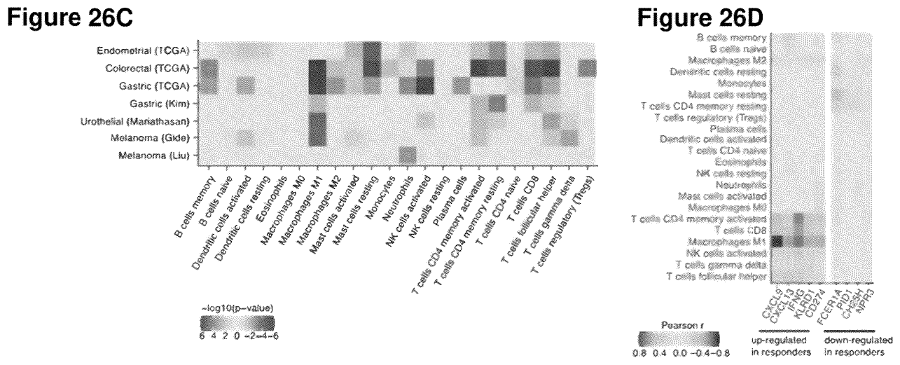

Hereinafter, embodiments of the present computer-implemented system and method for predicting clinical response of a tumor to immune checkpoint blockade therapy (ICB) will be described with reference to FIG. 1 to FIG. 25. It is to be understood that limiting the description to the preferred embodiments of the invention is merely to facilitate discussion of the present invention and it is envisioned without departing from the scope of the appended claims.

The disclosed embodiments relate to clinical decision support systems and computer-implemented methods for predicting clinical response of a target tumor to ICB. The embodiments comprise a response estimation model (also referred to as an IOSelect model/IOSelect clinical platform) to process sequenced transcriptome data from a cancer tissue sample and generate a prediction regarding potential impact/benefit of ICB. The data-driven clinical platform, IOSelect prioritized cancer patients for immunotherapy from molecular profiles of individual tumors. IOSelect is developed based on a gene signature identified based on analysis of transcriptome data of cancer tissue from known responders and non-responders to ICB. IOSelect uses multi-omics data and machine learning to predict cancer patients' potential to respond to ICB. IOSelect presents a comprehensive view of tumor's immune landscape, including a personalized score of predicted response from our multivariate model, in a user-friendly data dashboard. Rather than relying on single marker tests which are unable to capture the full complexity of tumors, the clinicians can make better informed treatment recommendations based on the interpretable machine learning platform (IOSelect).

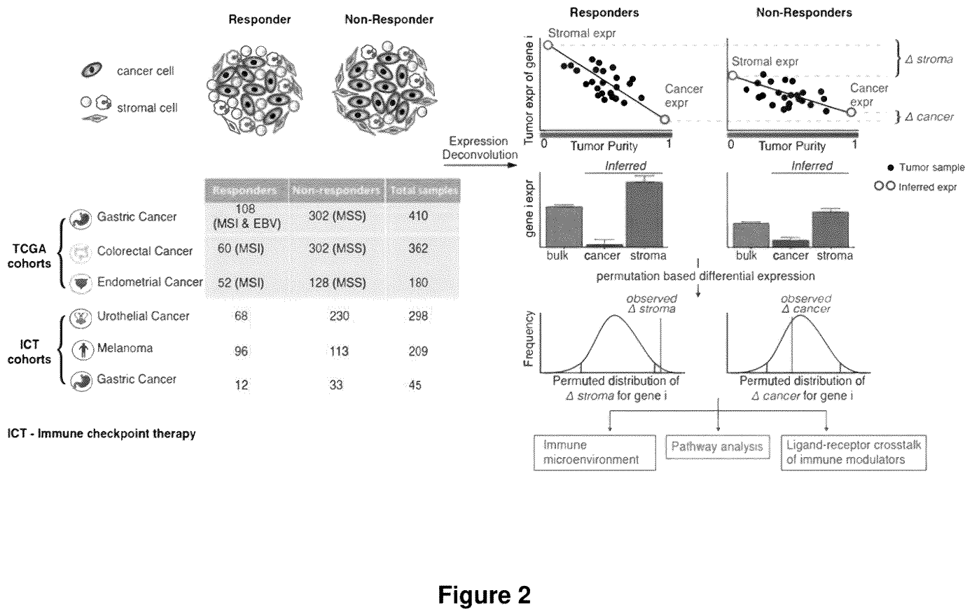

Since transcriptomic differences between MSI and MSS tumors may potentially predict tumor response to ICB, a meta-analysis of ICB response in 1486 tumors was performed. These tumor samples included transcriptomes of 534 pre-treatment tumors from ICB-treated patients comprising 4 studies of 3 tumor types (gastric cancer, urothelial cancer, melanoma), and 952 tumors from 3 cancer types in TCGA with the highest frequency of MSI tumors (colorectal cancer, gastric cancer and endometrial cancer as shown in FIG. 2. Using a deconvolution technique to estimate the stroma-specific and cancer-specific gene expression changes, common transcriptomic features of ICB response were identified across cancer types. A machine learning model (IOSelect model/algorithm) was built to predict patient response to ICB based on the novel biomarkers identified.

TABLE 1
Clinical characteristics of the study cohorts

| | | | cancer | | Type of | Response | | |
|---|---|---|---|---|---|---|---|---|
| cohort | type | PMID | type | ICB agent | ICB | R | NR | total |
| Mariathasan | discovery | 29443960 | bladder | atezolizumab | anti-PD- | 68 | 230 | 298 |
| | | | | | L1 | | | |
| Kim | discovery | 30013197 | gastric | pembrolizumab | anti-PD- | 12 | 33 | 45 |
| | | | | | 1 | | | |
| Liu | discovery | 31792460 | melanoma | nivolumab or | anti-PD- | 47 | 71 | 118 |
| | | | | pembrolizumab | 1 | | | |
| Gide | discovery | 30753825 | melanoma | pembrolizumab | anti-PD- | 40 | 33 | 73 |
| | | | | or nivolumab | 1 alone | | | |
| | | | | alone, or with | or with | | | |
| | | | | ipilimumab | anti- | | | |
| | | | | | CTLA-4 | | | |
| Riaz | validation | 29033130 | melanoma | nivolumab | anti-PD- | 10 | 39 | 49 |
| | | | | | 1 | | | |
| Pender | validation | 33020056 | lung (20), | not specified | PD- | 15 | 72 | 87 |
| | | | melanoma | | 1/PD-L1 | | | |
| | | | (18), | | (57), | | | |
| | | | breast | | CTLA-4 | | | |
| | | | (13), | | (3), | | | |
| | | | others | | combo | | | |
| | | | (36) | | (26), | | | |
| | | | | | others | | | |
| | | | | | (1) | | | |
| Freeman | validation | 35243413 | melanoma | not specified | PD- | 10 | 21 | 31 |
| | | | | | 1/PD-L1 | | | |
| | | | | | (13), | | | |
| | | | | | CTLA-4 | | | |
| | | | | | (15), | | | |
| | | | | | combo | | | |
| | | | | | (3) | | | |
| Jung | validation | 31537801 | lung | not specified | anti-PD- | 19 | 8 | 27 |
| | | | | | 1 or PD- | | | |
| | | | | | L1 | | | |
| Nathanson | validation | 27956380 | melanoma | ipilimumab | anti- | 12 | 7 | 19 |
| | | | | | CTLA-4 | | | |
| Ratovomanana | validation | 37269904 | colorectal | nivolumab alone | anti-PD- | 13 | 11 | 24 |
| | | | | or with | 1 alone | | | |
| | | | | ipilimumab | or with | | | |
| | | | | | anti- | | | |
| | | | | | CTLA-4 | | | |

Such a machine learning model is embodied by the methods used herein. Such a computer-implemented method 100 is reflected in FIG. 1 and used for predicting clinical response of a tumor to immune checkpoint blockade therapy (ICB). The method 100 involves generating a response estimation model (step 102) by:
(a) obtaining a training dataset;
(b) deconvolving training dataset to identify differentially expressed genes (DEGs);
(c) performing regression analysis of the features to select a subset of the features as most predictive of the responder or non-responder labels and obtain associated feature weights (step 102c);
(d) incorporating the subset of features and associated feature weights in the response estimation model (step 102d);
obtaining transcriptome data of a target tumor tissue sample (step 104); and
processing the transcriptome data using the generated response estimation model to generate an estimated response metric indicating a likely response of the target tumor to ICB (step 106).

### Analysis and Identification of Biomarkers of Immune Checkpoint Blockade Therapy (ICB) Response

Systematic analysis of potential biomarkers of ICB response is constrained by the small number of molecularly characterized tumors in most published ICB studies. In contrast, through systematic efforts of tumor sequencing in the past few decades, molecular data of large numbers of microsatellite instable (MSI) tumors are publicly available. Considering the large response differential between microsatellite instable (MSI) and microsatellite stable (MSS) tumors (almost half of MSI tumors respond to immune ICB, compared to 5% in MSS tumors), the embodiments leverage transcriptomic differences between MSI and MSS tumors that have the potential to be generalized to predict tumor response to ICB. The embodiments leveraged on (1) large the cancer genome atlas (TCGA) cohorts with high MSI prevalence and (2) tumors from patients treated with ICB in a combined analysis to identify features predictive of ICB response. A meta-analysis was performed in some experiments on the transcriptomes of 534 tumors from 4 ICB cohorts of 3 cancer types (gastric cancer, urothelial cancer and melanoma), and 952 tumors from 3 TCGA cancer types with the highest frequency of MSI tumors (gastric cancer, colorectal cancer and endometrial cancer).

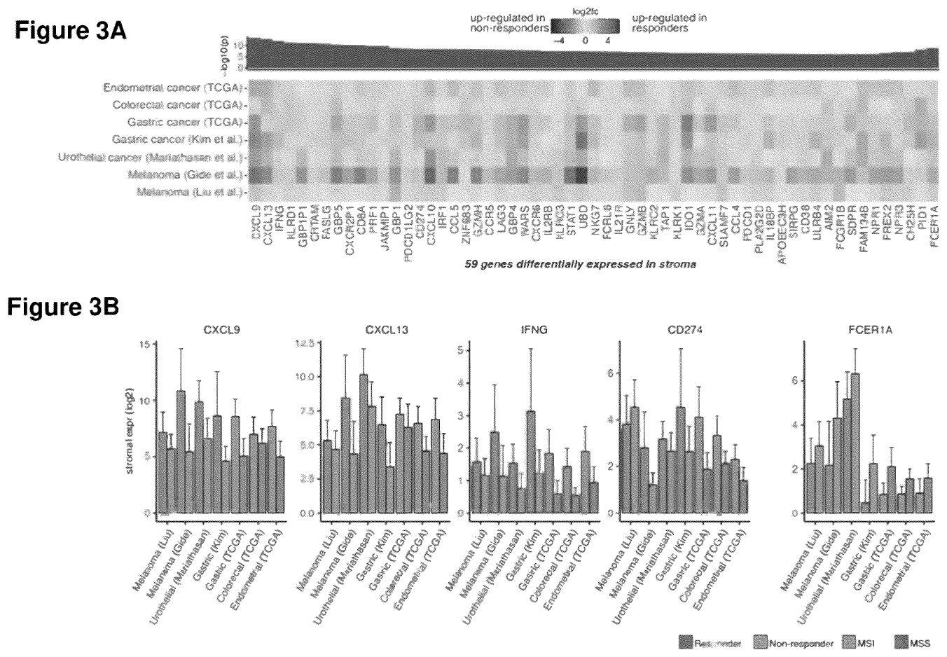

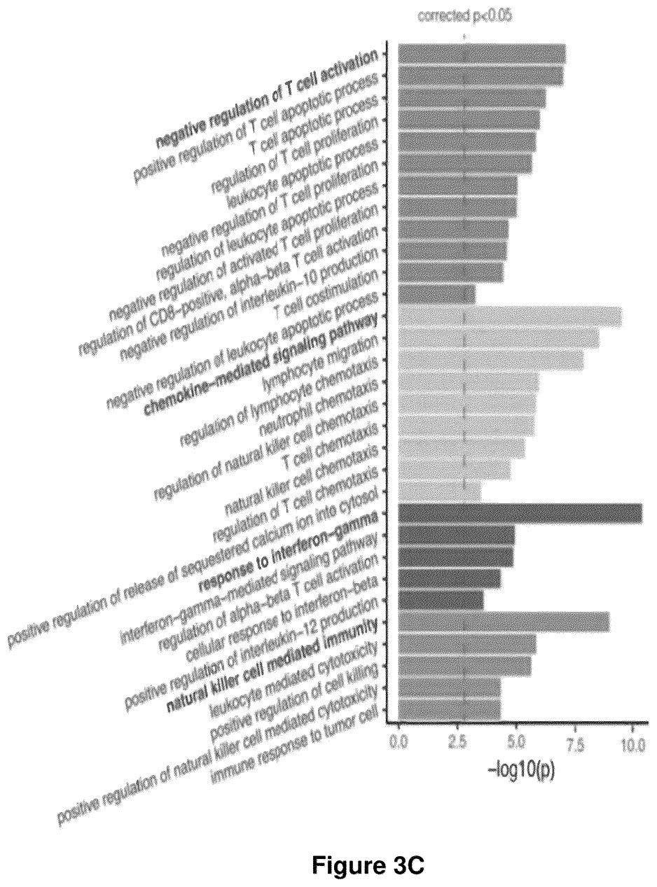

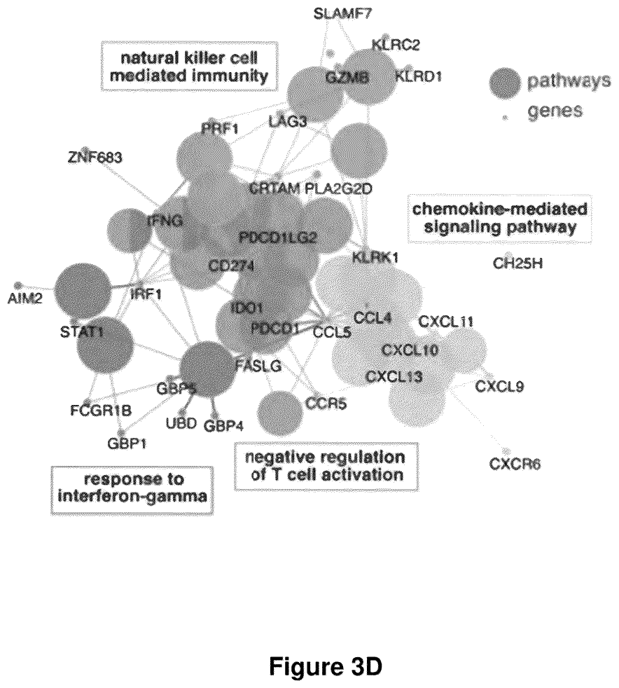

Tumors are a heterogenous masses of cancer cells, and non-cancerous stroma cells including immune cells, fibroblasts and endothelial cells. Response to immunotherapy is dependent on an interplay of multiple co-stimulatory and co-inhibitory interactions between effector cells, antigen-presenting immune cells such as dendritic cells and macrophages, as well as cancer cells in the tumor micro-environment (TME). Dysregulation of these ligand-receptor interactions, or immune checkpoints, leads to immunosuppression in the tumor. However, it is challenging to tease apart these complex interactions between cancer and immune cells using bulk tumor transcriptomics. The heterogenous nature of the TME can confound molecular signature discovery. To tease apart the signals from the cancer cells and stroma cells, expression deconvolution methods were developed and applied to infer differentially expressed genes (DEGs) from a plurality of candidate genes. In some embodiments the DEGs were identified separately in the cancer and stroma compartments separately. Experiments demonstrated that the most consistent differential expression signals were from the stroma but not the cancer compartment. This suggests that cancer intrinsic features of ICB response tend to be cancer type specific, while stroma intrinsic features are more generalizable to other cancer types and therefore are more suitable biomarkers for ICB response prediction. From this analysis of the experimental results, 59 stromal genes were identified that are consistently differentially expressed between responders (MSI) and non-responders (MSS) across cancer cohorts as shown in FIG. 3A.

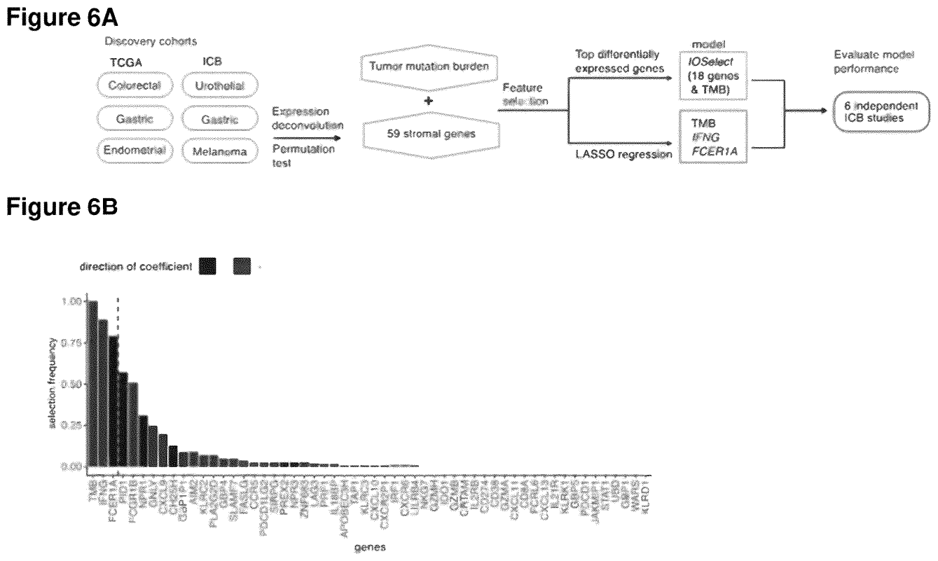

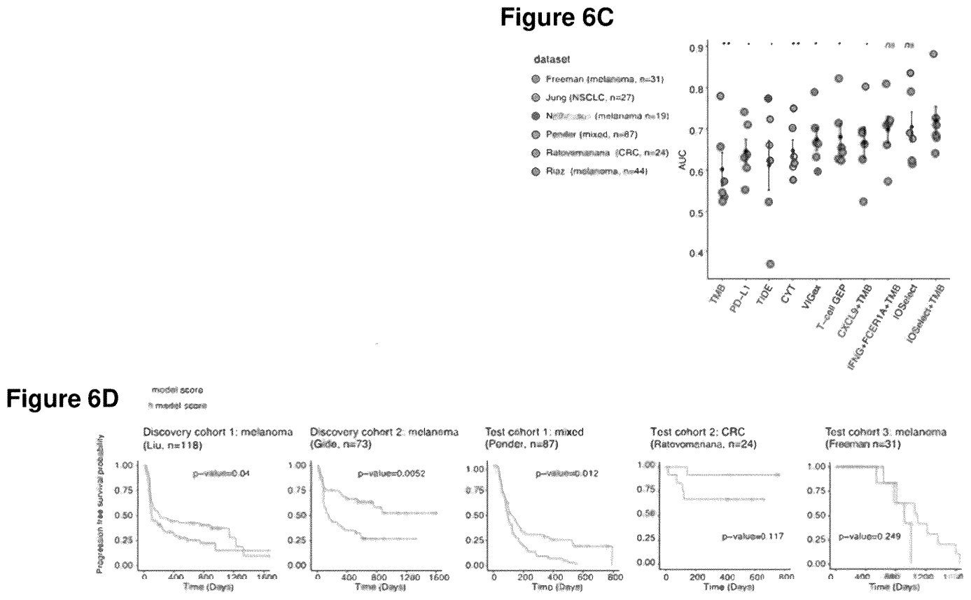

To build a predictive model of ICB response, various feature selection strategies and machine learning methods were evaluated, including least absolute shrinkage and selection operator (LASSO) regression, step-wise regression, random forest, gradient boosting and support vector machine (SVM). LASSO regression appeared to be the best performing method over the available data, however other regression methods may also be applicable on different datasets. As described in step 102a, a training dataset is produced from training transcriptome records of tumor tissue samples of a plurality of known responders and a plurality of known non-responders to ICB. A response estimation model or an IOSelect model is generated from 534 tumors with clinically annotated ICB response (responder or non-responder labels) used as training data. Robust selection of the most informative features of ICB response were then identified experimentally, using LASSO logistic regression. In some embodiments, an IOSelect model was built using 4 selected genes. Other experiments using different training dataset may result in the selection of a different set of genes. Next, experiments were performed to evaluate model performance on the training cohorts (using 10-fold cross validation) and 2 independent test cohorts. Test cohort 1 consisted of 49 tumors from melanoma patients treated with PD-L1 inhibitor nivolumab. Test cohort 2 consisted of 87 tumors from 20 solid cancer types treated with a mix of ICB agents. The areas under the receiver operating curve (AUC) of the IOSelect model was calculated and benchmarked against existing biomarkers of ICB response, namely tumor mutational burden (TMB), PD-L1 expression and T-cell gene expression profile (GEP) as shown in FIG. 6C and 16.

The present response estimation model significantly outperforms existing biomarkers in most cases. The 4-gene signature in the IOSelect model is a measure of the favorability of the tumor microenvironment while TMB is a measure of tumor immunogenicity where increased mutation load is associated with higher neo-antigen burden and anti-tumoral immune responses. However, the predictive power of TMB varies significantly by cancer type and it is difficult to define a single TMB threshold across cancer types. They thus represent different facets of the anti-tumoral immune response, and therefore are likely complementary biomarkers. The IOSelect multi-omic model of some embodiments combined both the 4-gene signature and TMB, and showed that the IOSelect multi-omic model has the best performance for all cohorts except for test cohort 1, where the IOSelect RNA only model performed slightly better than the multi-omic model. Overall, the IOSelect multi-omic model outperforms PD-L1 expression by 15-40% as illustrated in FIG. 6C and 16.

A clinical software platform/clinical decision support system according to the embodiments allows prioritization of treatment of cancer patients for immunotherapy. The platform streamlines the pre-processing, quantification/mutation calling on transcriptomic and genomic data, using state-of-art bioinformatics pipeline. New patient data (data comprising transcriptome sequences) may be normalized against existing cohorts to remove potential biases from batch effect and technological differences. Then, the IOSelect algorithm outputs the predicted probabilities of ICB response using the RNA-only model and/or the multi-omics model, depending on data availability. To provide a more comprehensive view of patient's potential to respond to ICB, IOSelect may calculate the percentile scores of published biomarkers, such as TMB, PD-L1 expression and T-cell GEP, to be used in clinical discussions as illustrated in FIG. 16.

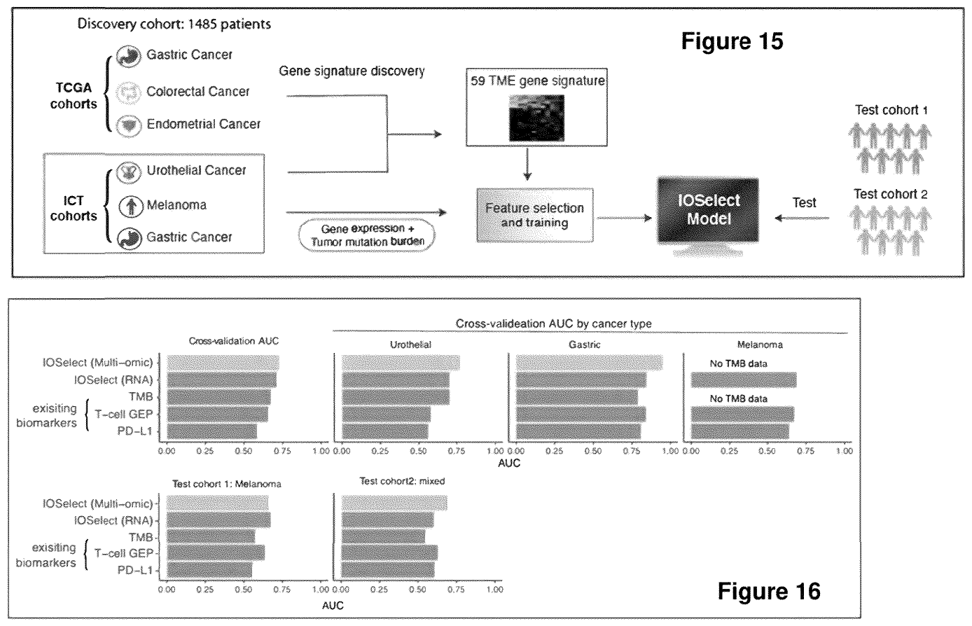

If whole exome-sequencing is performed, the present response estimation model of some embodiments also demonstrates clinically actionable genomic mutations associated with immunotherapy, such as V-Raf Murine Sarcoma Viral Oncogene Homolog B (BRAF) mutations in melanoma and epidermal growth factor receptor (EGFR) mutations in lung cancer. Individual molecular features of each tumor and the contributions of each feature to the final score are shown in FIG. 15.

Currently, PD-L1 expression is the most widely adopted biomarker to guide clinical decision making on ICB treatment, but single marker tests like PD-L1 immunohistochemistry assays (IHC) are unlikely to capture the full complexity of tumors. T-cell GEP is another popular proposed biomarker of ICB response that measures the degree of T-cell infiltration based on gene expression profile. The multi-omics approach of the present response estimation model combining an identified gene signature with TMB can better capture the multi-faceted tumor immune landscape and therefore has superior performance compared to existing biomarkers. Currently, clinical adoption of sequencing-based assays of immunotherapy response is limited by the lack of technical solutions that can efficiently process, interpret and present large quantitative datasets to clinicians. The proposed clinical platform is directed at addressing these technical hurdles by streamlining the data processing, analysis and response prediction. It presents a comprehensive view of tumor's immune landscape, including a personalized score of predicted response from a multivariate model, together with other complementary biomarkers. The present response estimation model thus facilitates a rapid turnaround time from sample collection to data presentation.

### Tumor Transcriptome Deconvolution to Identify Common Features of ICB Response in Cancer and Stromal Cells

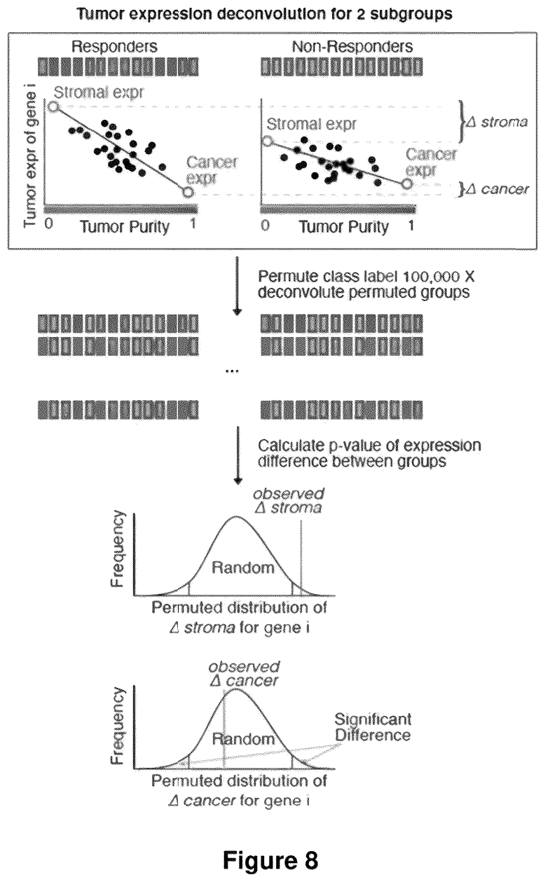

Step 102b of FIG. 1 involves performing deconvolution of the training dataset to identify differentially expressed genes (DEGs) in the training dataset. In so doing, the DEGs are treated as features of the training dataset and associated responder, non-responder status is treated as a label of the records in the training dataset. In some embodiments, transcriptomic deconvolution was performed to estimate the stromal- and cancer-cell expression of each gene in tumors from responders (MSI) and non-responders (MSS) separately as shown in FIG. 2. As Epstein-Barr virus-(EBV)-positive gastric tumors are also enriched in ICB responders, the MSI and EBV tumors were grouped together as the potential responder group for gastric cancer. In some experiments, the tumor purity of each sample was calculated based on transcriptomic and/or genomic data (where available). Then, for each response group, a negative least squares regression of gene expression against tumor purity may be performed. This is done to estimate the mean expression in the stromal and cancer cells for each gene per step 102b. After quantifying the observed expression difference of each gene between responders and non-responders in both cancer and stromal cells, differentially expressed genes (DEGs) are identified using a permutation based statistic as disclosed in FIG. 2 and FIG. 8.

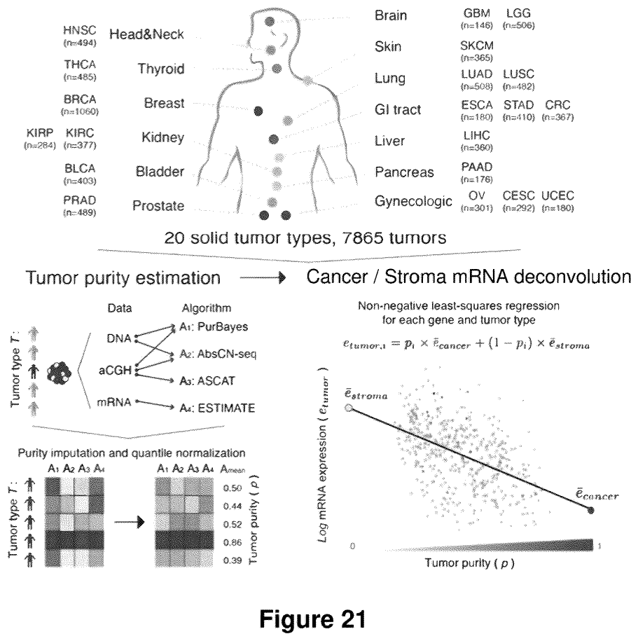

In some embodiments, the first step in deconvolution is to determine the proportion of cancer cells in a tumor sample. This may be achieved using a combination of approaches that make use of either somatic variant allele frequency or B-allele frequency data (obtained from WGS or Exome-seq data) to estimate the ploidy and purity of a given tumor sample. Since estimates usually vary considerably across individual algorithms, and algorithms sometimes fail to converge, some embodiments use a combination of missing data imputation and quantile normalization to obtain robust estimates of tumor purity as shown in FIG. 21. A second step of some embodiments aims to construct a deconvolution approach to separate transcriptome profiles of cancer, immune, and other stromal cells. This step is directed at robustly estimating the relative proportions of immune and stromal cells in a tumor. Some embodiments achieve this using transcriptional signatures to estimate relative proportions of these two cell-type supersets in tumors. With these three relative cell-type proportions estimated from the tumor genomic and transcriptomic data, a constrained regression approach can be applied to estimate mean gene expression levels for a given gene in each cell-type across a set of tumors. For a given gene, the constrained regression approach can be calculated using the formula:

$${e}_{tumor , i} = {p}_{cancer , i} \times {\overline{e}}_{cancer} + {p}_{immune , i} \times {\overline{e}}_{immune} + {p}_{stroma , i} \times {\overline{e}}_{stroma} with {p}_{cancer , i} + {p}_{immune , i} + {p}_{stroma , 1} = 1$$

Some embodiments make the simplifying assumption that these (non-negative) average cell-type expression levels are constant across the set of tumors and estimate them using non-negative least squares regression. Experiments demonstrated that the equation above tends to overestimate stromal gene expression for genes with somatic copy number alterations (CNA) affecting gene expression in a subset of the samples (for example ERBB2 in HER2-positive breast tumors). Some embodiments therefore use a modified approach for such genes. These embodiments first identify genes with correlation between CNA and mRNA expression (comparing expression for samples with diploid and non-diploid CNA, Mann-Whitney U-test, P<1e-6, to account for multiple testing) in a given set of tumors, and then estimate cancer and stromal compartment gene expression. The estimation is performed using a two-step approach. Stromal compartment mRNA expression is first inferred using the above approach using only samples with diploid copy number for the gene. Embodiments then use the inferred mean stroma compartment expression, the measured mean tumor expression, and the mean purity of the tumor samples to calculate the mean cancer compartment expression using the above equation. Cancer/stromal-cell differentially expressed genes (DEGs) between ICB responders and non-responders are identified using a permutation based statistic as shown in FIG. 2 and FIG. 8.

Top Stromal Genes Associated with ICB Response are Enriched in Immune Pathways

From this unbiased, transcriptome-wide analysis, 59 consensus DEGs are identified in the stroma as shown in FIG. 3A, 3B, FIGS. 26A and 26C among which 51 are genes with immune-related functions, including genes encoding checkpoint proteins (e.g. CD274, LAG3, CTLA4), and genes involved in interferon gamma signaling (e.g. IFNG, IRF1, STAT1), T-cell effector functions (e.g. PRF1, GZMA, GZMB), and NK-cell mediated cytotoxicity (KLRD1, KLRC2). The top 2 DEGs with the greatest overexpression in ICB responders are chemokines CXCL9 and CXCL13. CXCL9 is a key chemokine responsible for T-cell trafficking through binding its receptor CXCR3 on T-cells. It is associated with increased T-cell infiltration into tumors. CXCL13 is involved in both B-cell and T-cell migration, mediated through its receptor CXCR5. Production of CXCL13 by T follicular helper cells (TFH) is essential for tertiary lymphoid structure formation at tumor site and germinal center B cell activation, which is associated with anti-tumoral response to ICT. Several recent studies proposed CXCL9 and CXCL13 to be predictors of improved ICB response. Remarkably, these two chemokines were also highlighted by a recent meta-analysis that evaluated existing biomarkers of ICB response, suggesting that CXCL9 and CXCL13 are indeed the strongest individual transcriptomic predictors of ICB response independent of tumor type.

Stromal Genes Negatively Associated with ICB Response

In some experiments, a group of immune-related genes that are negatively correlated with ICB outcome were also observed. FCER1A demonstrated the most consistent and greatest overexpression in ICB non-responders. FCER1A encodes a subunit of the IgE receptor. While it is an established marker of basophils, mast cells and dendritic cells, it has also been shown to be expressed on the immunosuppressive M2 macrophages and tumor associated macrophages (TAMs). CH25H, a gene encoding enzyme cholesterol 25-hydroxylase that catalyzes the formation of 25-hydroxycholesterol (25-HC), is also up-regulated in ICB non-responders. The oxysterol 25-HC is produced by macrophages in response to type I interferon signalling, and has numerous immunological effects including B-cell chemotaxis, macrophage differentiation and regulation of inflammatory response. Two (2) natriuretic peptides NPR1 and NPR3 among genes up-regulated in ICB non-responders were also observed. Although natriuretic peptides function primarily to regulate electrolytes, they are expressed in immune cells and there is increasing evidence of their role in inflammation and immunity.

Next, analysis was performed on the biological pathways enriched among DEGs associated with ICB response. Based on the analysis, 31 enriched Gene Ontology biological processes terms were identified, all of which are immune related as disclosed in FIG. 3C. FIG. 3D shows that the 31 terms can be further grouped in to 4 clusters based on similarity. Overall, genes associated with ICB response are enriched in T cell activation, chemokine signaling, interferon-gamma response, and natural killer cell mediated cytotoxicity.

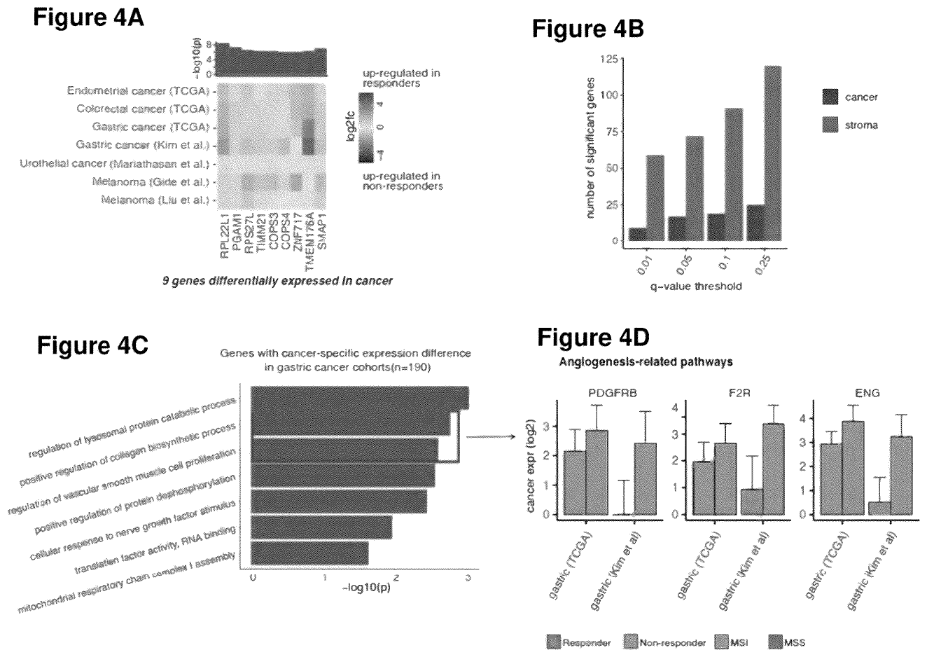

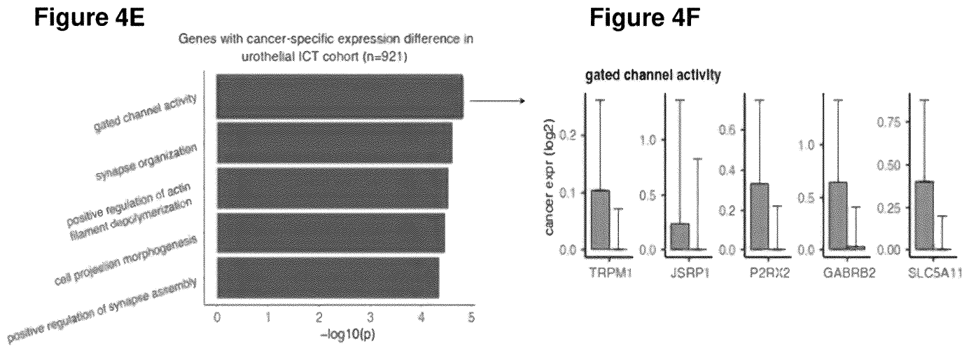

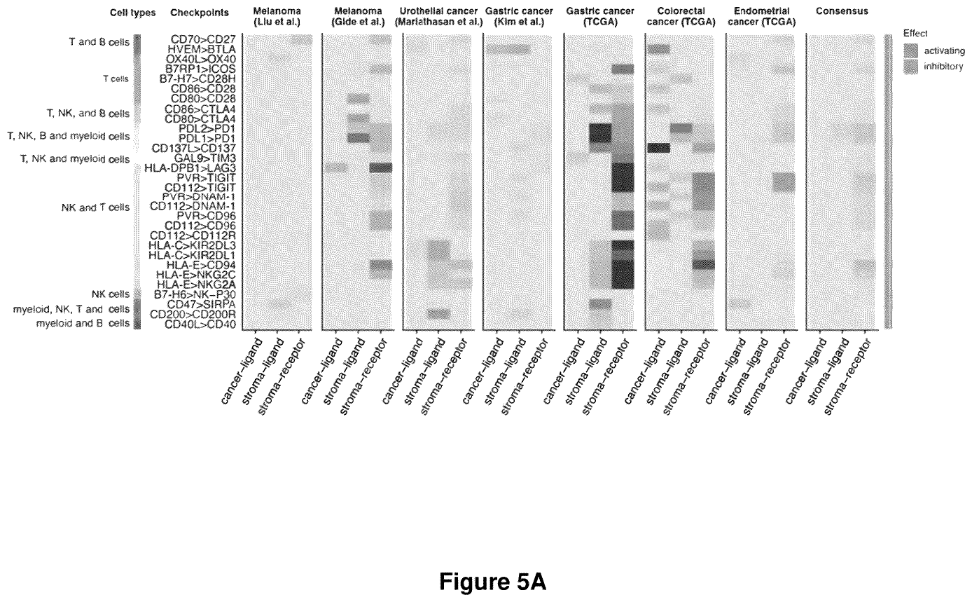

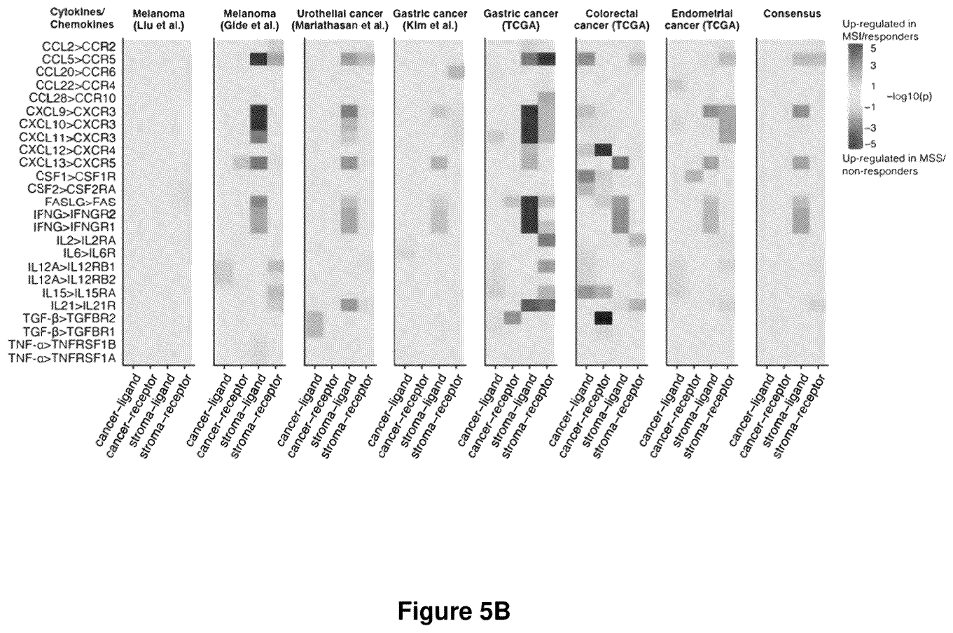

In cancer cells, the experiments identified 9 genes differentially expressed between ICB responders and ICB non-responders as shown in FIG. 4A, 9B and 9D. However, the differential expression signal was the strongest and most consistent among the TCGA cohorts and the gastric cancer ICB cohort, where all responders are of the MSI subtype. Therefore, the DEGs identified in the cancer cells are likely to be related to MSI biology, but not specific to ICB response. In general, fewer cancer specific consensus DEGs were observed compared to stroma specific consensus DEGs, regardless of the q-value cutoff as disclosed in FIG. 5B. Furthermore, no functional enrichment is found even at a lower q-value cutoff of 0.25. The lack of consensus DEGs across cancer types suggested that cancer intrinsic differences associated with ICB response tend to be cancer type specific.

Next, pathway enrichment analysis was performed on individual cancer types to identify potential cancer-specific processes involved in ICB response. For melanoma, the experiments only identified 22 DEGs shared between the 2 melanoma cohorts, and did not find any enriched pathways. In gastric cancer, the experiments found 190 consensus DEGs between the gastric ICB cohort and TCGA STAD cohort. Interestingly, FIG. 4C discloses identified 2 groups of angiogenesis related functional clusters, “positive regulation of collagen biosynthetic process” and “regulation of vascular smooth muscle cell proliferation” that are up-regulated in ICB non-responders. FIG. 4D shows pro-angiogenic genes ENG, F2R and PDGFRB are overexpressed in ICB non-responders (MSI/EBV) compared to responders (MSS). The results were consistent with the understanding that angiogenesis is associated with immune suppression, and suggests that a pro-angiogenic environment is associated with ICB resistance in gastric cancer. In urothelial cancer, cancer specific DEGs are enriched in neuron-related functional terms as shown in FIG. 4E. Neuronal genes were overexpressed in ICB responders compared to ICB non-responders as shown in FIG. 4F. This supports previous finding that the neuronal TCGA expression subtype of urothelial cancer is associated with poor survival but high response rate to ICB.

### Single-Cell RNAseq Confirms Differential Expression of the Stromal Biomarkers in Specific Immune Cell Subsets

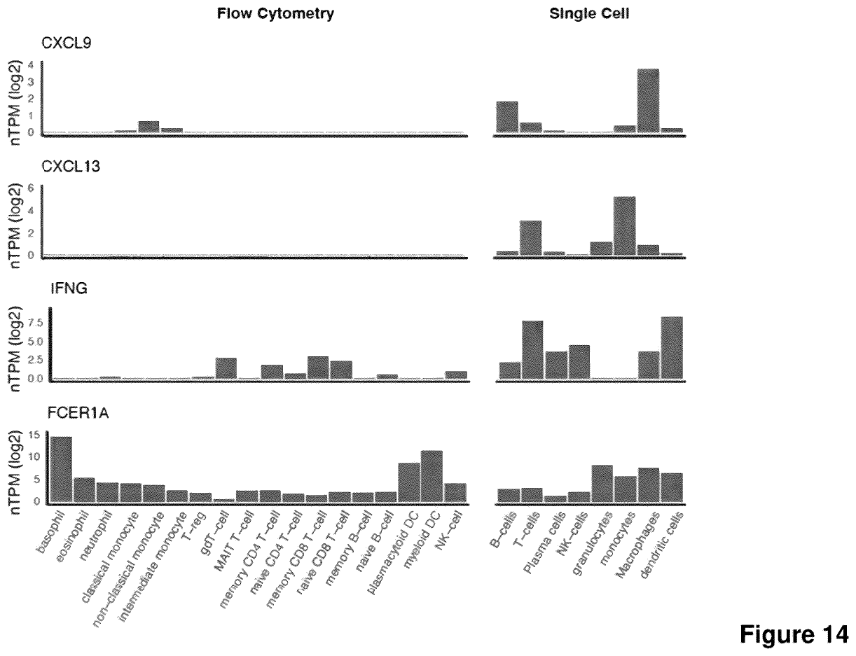

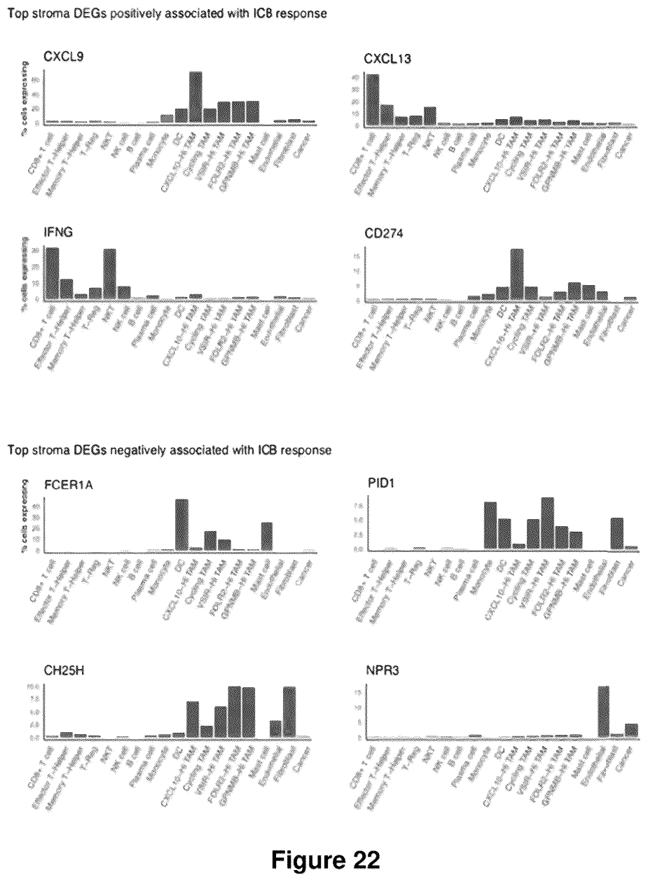

The expression of the stromal DEGs in specific stromal cell subsets in connection to ICB response is studied using a single-cell RNAseq (scRNAseq) dataset of patients with renal cell cancer treated with ICB. Stromal (non-cancerous) cells express the stromal DEGs differently in the predicted direction between ICB responders and non-responders as shown in FIG. 26A. Then, the stromal DEGs' expression distribution among individual type of cell is examined. It was discovered that CD8+T-cells are enriched in CXCL13 and IFNG, whereas M1-like tumor-associated macrophages (TAMs) express the highest levels of CXCL9 and CD274 as shown in FIG. 22. PID1 and CH25H are enriched in myeloid cells and fibroblasts, while NPR3, NPR1, PREX2, and SDPR are exclusively expressed by endothelial cells. Of the DEGs linked to poor ICB response, FCER1A is most frequently expressed by dendritic cells, mast cells, and M2-like TAMs, also shown in FIG. 26A. As a result, stroma DEGs include several indicators of the tumor microenvironment, such as abundance of fibroblasts, T-cell infiltration, macrophage polarization, and angiogenesis. The cell-type specific expression of the top stromal DEGs is confirmed using orthogonal expression data of immune cell subtypes from flow cytometry and scRNAseq experiments from the Human Protein Atlas, shown in FIG. 14. Moreover, it is discovered that the increased abundance of specific immune cell subsets contributes to the differential gene expression signal between ICB responders and non-responders. For instance, ICB responders have higher frequencies of CXCL9+M1-like TAMs and CXCL13+ T cells than non-responders do, whereas non-responders have higher frequencies of FCER1A+M2-like TAMs and FCER1A+dendritic cells, as shown in FIG. 26B. These findings imply that some elements of the TME, such as the stromal DEGs, are responsible for determining the tumor's ICB sensitivity.

### Immune Cell Subtype Analysis Identifies M1 Macrophages as Strong Correlate of ICB Response

Immune cell composition of the TME is a determinant of immunotherapy response. The experiments estimated the immune cell composition of each tumor using Cibersortx and compared the abundance of immune cell subsets between ICB responders and non-responders. It is found that macrophage M1, T-follicular helper (Tfh), and activated CD4 memory T-cells to be to the most enriched in ICB responders compared to non-responders as disclosed in FIG. 26C and FIG. 12. Macrophage M1 is known to be pro-inflammatory and anti-tumoral. The results further demonstrate that M1 macrophage infiltration could be a general feature of

ICB response across multiple cancer types. Tfh cells are specialized CD4+ T cells that aid the formation of germinal centers in tertiary lymphoid structures (TLS). They interact with B-cells at TLSs to activate antibody responses from B cells and enhance CD8+T-cell effector functions. The presence of Tfh cells are associated with favorable survival in breast, lung and colorectal cancers. However, their involvement in ICB response has not been well established. Our analysis also demonstrated that while resting CD4 memory T-cells were enriched in non-responders, activated CD4 memory T-cells were enriched in ICB responders. This suggests that pre-existing CD4+T-cell immunity is important for effective anti-tumoral response upon ICB treatment.

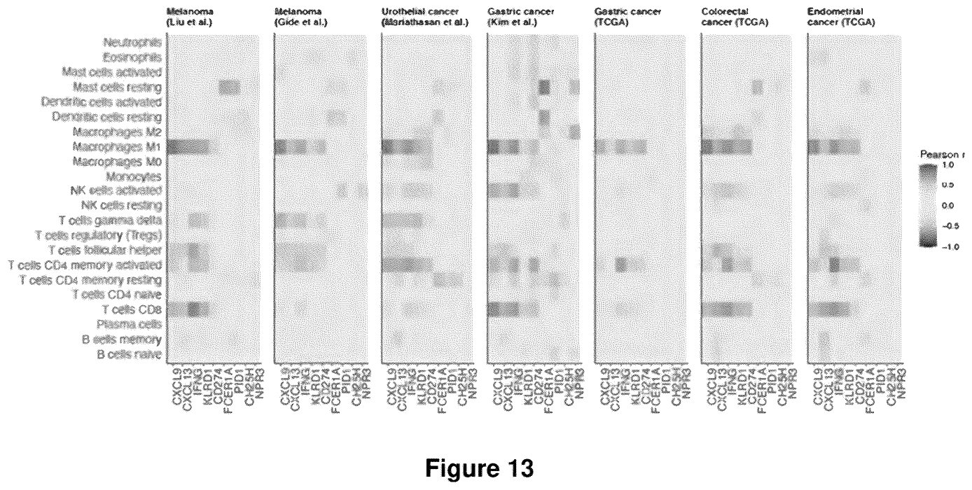

Next, the top N (N is, e.g., 10 or some other desired number) DEGs associated with ICB response where analyzed to assess whether they represent differential infiltration of specific immune cell subsets. The experiments found that the top gene markers of ICB response are associated with abundance of M1 macrophages, CD4+ and CD8+T-lymphocytes, and activated NK cells as shown in FIG. 26D and FIG. 13. In contrast, the DEGs associated with poor ICB response, including FCER1A, were correlated with resting state of mast cells, CD4+ memory T cells, and dendritic cells, suggesting that these genes are likely markers of a quiescent or immune-suppressive microenvironment, also as shown in FIG. 26D and FIG. 13.

### Cancer Intrinsic Features of ICB Response Tend to be Tumor Type Specific

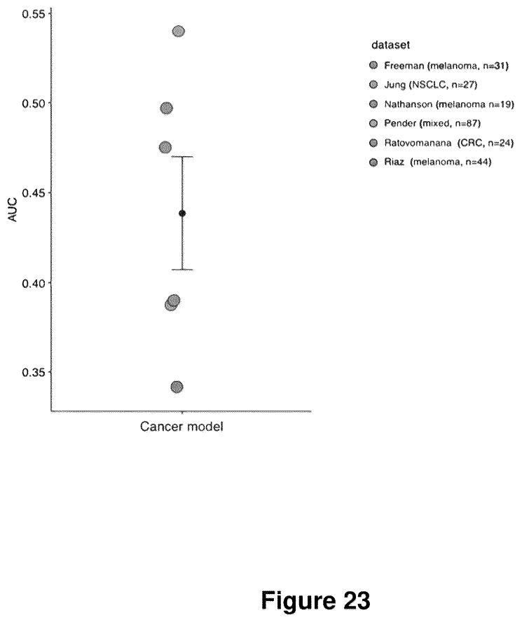

Previous studies have proposed multiple mechanisms by which cancer cells may directly suppress tumor immunity and impede ICB therapy. Surprisingly, our meta-analysis across tumor types revealed a paucity of DEGs associated with ICB response in cancer cells. In cancer cells, we identified 9 genes differentially expressed between ICB responders and ICB non-responders across tumor types as shown in FIG. 4A and FIG. 26B & D. However, we observed that the differential expression signal for these 9 genes was mostly driven by the MSI ICB-responder-enriched cohorts. As compared to stromal DEGs shown in FIG. 3A, the signal for the cancer DEGs was less consistent across the distinct ICB cohorts shown in FIG. 4A. Therefore, it is hypothesized that the cancer DEGs identified by the meta-analysis are likely related to biology of MSI tumors, rather than general mechanisms of ICB response. Indeed, the 8 cancer DEGs are not predictive of ICB response when tested on tumor transcriptomic data from 6 independent ICB studies of FIG. 23. It is also confirmed that the paucity of cancer cell DEGs, as compared to stroma cell DEGs, persisted regardless of the significance cutoff in our meta-analysis shown in FIG. 4B, and functional enrichment or relationships among cancer DEGs cannot be identified.

Next, the same DEG analysis is conducted within individual tumor types to explore the potential for tissue-dependent processes involved in ICB response. For melanoma, only 22 cancer DEGs shared between the 2 melanoma cohorts are identified, and these genes were not enriched in specific pathways. In gastric cancer, 190 cancer ICB response DEGs are identified across the gastric ICB cohort and TCGA cohort. Interestingly, genes associated with poor ICB response were enriched for angiogenesis-related functions, as shown in FIG. 4C, with pro-angiogenic genes ENG, F2R and PDGFRB overexpressed in ICB non-responders (MSI/EBV) compared to responders (MSS), shown in FIG. 4D. These data are consistent with the understanding that angiogenesis can be associated with immune suppression, suggesting that a pro-angiogenic environment may impede ICB response in gastric cancer. In urothelial cancer we found that cancer DEGs were enriched for neuron-related functional terms, shown in FIG. 4E, with neuronal genes overexpressed in ICB responders compared to ICB non-responders shown in FIG. 4F. This observation is concordant with a previous study linking the neuronal expression subtype of urothelial cancer with poor overall survival but high response rate to ICB.

Overall, while these results indicate an overall paucity of tissue-agnostic cancer-cell-intrinsic features of ICB response, they also highlight the potential for tissue and tumor-type specific expression signatures in cancer cells that may predict ICB response.

### Lack of Cancer Intrinsic Immune Checkpoint Regulation Across Cancer Types

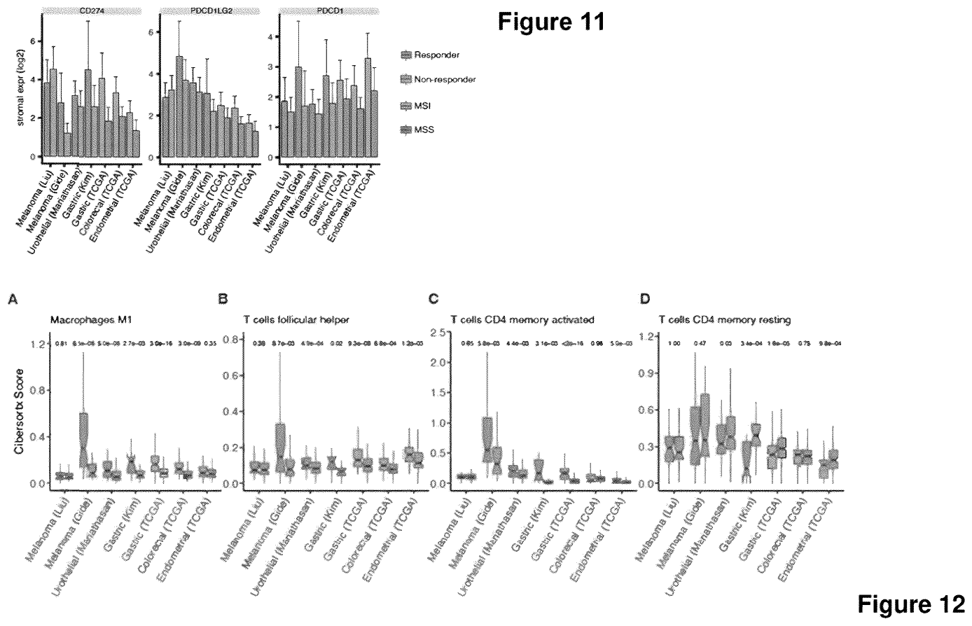

Although the transcriptome-wide analysis failed to identify conserved cancer-specific DEGs of ICB response, experiments were conducted to examine if the cross-talk between immune checkpoint ligands and receptors between cancer and immune cells could promote primary ICB resistance. Specifically, the expression patterns of 30 known immune checkpoint ligand-receptor pairs in both the cancer and stromal components of the TME is examined, to identify ligand-receptor pairs with coordinated expression changes between ICB responders and non-responders. In this targeted analysis, all ligand-receptor genes with nominal p-value >0.1 from the differential gene expression permutation test are surveyed to uncover more subtle signals that might have been missed in the transcriptome-wide analysis. It is found that stromal overexpression of 13 checkpoint receptors and 2 checkpoint ligands to be associated with ICB response across cancer types as shown in FIG. 5A. Checkpoint ligands PD-L1 and PD-L2 and their receptor PD-1 were coordinately upregulated in the stroma of ICB responders compared to ICB non-responders. PD-L1 (CD274) and PD-1 were significantly upregulated in the stroma of ICB responders in 6 out of 7 cohorts studied (p-value<0.05, permutation test), while PD-L2 was upregulated in 5 out of the 7 cohorts (shown in FIG. 11). Interestingly, no checkpoint ligands were consistently differentially expressed and associated with ICB response in cancer cells across tumor types.

In addition to immune checkpoints, cytokines also play important roles in tumor immunity. The expression of 13 pairs of cytokines and cytokine receptors known to be involved in tumor immunity is examined, aiming to identify cytokine signals associated with ICB response, as shown in FIG. 5B. Similar to the analysis for immune checkpoints, cancer-intrinsic cytokine expression was not associated with ICB response across tumor types. However, six cytokines (IFNG, FASLG, CXCL13, CCL5, CXCL10 and CXCL9), and the corresponding receptors for three of the cytokines (CCR5 for CCL5, CXCR3 for CXCL9 and CXCL10) were significantly upregulated in the stroma of ICB responders, consistent with previous observations. Overall, our analysis finds no evidence for a universal mechanism by which cancer cells inhibit immune cells via checkpoints interactions or chemokine/cytokine signaling.

### a Multivariate Classifier Robustly Predicts ICB Response Across Cancer Types

Next, experiments were performed to validate if the consensus DEGs identified earlier could improve the prediction accuracy of ICB response as shown in FIG. 6A. The experiments focused on the 59 stroma-specific DEGs as illustrated in FIG. 3A as they are more consistent across cancer types compared to cancer-specific DEGs, and are therefore more likely to be generalizable to other cancer types. To capture the balance of immune-activating and suppressive signals the TME, the IOSelect immune score is developed, which is the ratio of the mean expression of the top 10 DEGs associated with ICB response to the mean expression of all 8 DEGs associated with ICB resistance.

While the stromal transcriptomic signature is a measure of the favorability of the tumor microenvironment, TMB is a measure of tumor immunogenicity. As the stroma DEGs and TMB represent different facets of tumor immunity, they are likely orthogonal predictors of ICB response. Therefore, a multivariate classifier, IOSelect or a clinical decision support system to predict clinical response of a target tumor to immune checkpoint blockade therapy (ICB) is developed, using both the IOSelect immune score and TMB as input features. The IOSelect classifier was trained on 461 samples from the ICB discovery cohorts with both transcriptomic and genomic data. The model performance is evaluated on 6 independent test cohorts treated with ICB, comprising 232 patients with melanoma, colorectal, lung, and other cancers. The area under the receiver operating curve (AUC) of our model is calculated, and benchmarked against established biomarkers of ICB response, namely CXCL9+TMB, T-cell GEP, VIGex, cytolytic score, TIDE, TMB alone, and PD-L1 expression. The IOSelect classifier achieved a mean AUC of 0.72, significantly outperforming existing biomarkers with mean AUCs ranging from 0.60 (TMB alone) to 0.68 (T-cell GEP) as shown in FIG. 6C.

Next, evaluation is done to decide if simpler model comprising just the top few predictive features could be sufficient to robustly predict ICB response. To identify the most predictive features of ICB response, embodiments used LASSO logistic regression with 10-fold cross-validation to robustly select features from the set 59 stromal DEGs and TMB as disclosed in FIG. 6A. In some embodiments, the response estimation model was build using the top 3 features selected by LASSO: TMB, IFNG and FCER1A expression as shown in FIG. 6B. A simple 3-feature model attained a mean AUC of 0.70 across the 6 test cohorts, which is only marginally lower than the AUC attained by the full IOSelect model shown in FIG. 6C, p-value=0.40 by paired t-test). Furthermore, the 3-feature classifier is predictive of progression free survival (PFS) in cohorts with survival data available shown in FIG. 6D.

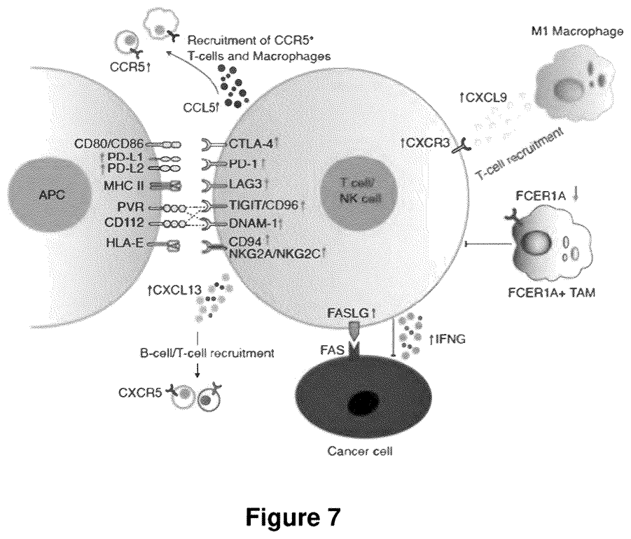

Using in-silico tumor transcriptome deconvolution, the mean cancer and stroma expression of each gene separately for the responder/MSI and non-responder/MSS tumors in each cancer cohort was estimated. A set of DEGs shared among multiple cancer types were then identified. The experiments found 59 stroma-specific consensus DEGs, which are strongly enriched in immune-related pathways and processes shown in FIG. 7. The top 2 stromal genes overexpressed in ICB responders are CXCL9 and CXCL13. Their roles in anti-tumor immunity and association with favorable ICB response have been demonstrated by multiple recent studies.

Previous studies suggest CXCL13 may play dual roles in ICB response. First, CXCL13 is produced by Tfh cells and involved in B-cell organization in TLS, and the presence of TLS can be a positive predictor of ICB response. Second, CXCL13 has been reported to be highly expressed by neo-antigen reactive CD8+T-cells directly involved in cancer cell killing. In line with these findings, we found enrichment of both Tfh and CD8+T-cells in tumors of ICB responders compared to non-responders across cancer types (shown in FIGS. 26 and 7). Additionally, the abundance of these two cell types correlates with CXCL13 expression. In agreement with recent work, we found that CXCL9 expression is strongly correlated with M1 macrophage infiltration, and M1 macrophages were one of the most differentially enriched immune cell types in ICB responders as shown in FIG. 26A. Although myeloid infiltration has traditionally been associated with immune suppression, our data has shown to be consistent with the hypothesis that M1 macrophage infiltration, and macrophage polarization, could be an important determinant of tumor sensitivity to ICB.

Further, a set of immune-related genes that were negatively associated with ICB response have been identified. Among these genes, FCER1A and CH25H have previously been reported to be over-expressed in anti-inflammatory M2 macrophages, and FCER1A+TAMs have been reported to promote tumor progression by engaging in a positive feedback loop with tumor initiating cells.

An enrichment of the resting states of CD4 memory T-cells, dendritic cells and mast cells in ICB non-responders have been observed. In contrast, the activated states of these cell types are enriched in the tumors of ICB responders as shown in FIG. 26B. Furthermore, while FCER1A is expressed in antigen presenting cells like dendritic cells and macrophages, it is noted that its expression is 25 times higher in resting dendritic cells compared to activated dendritic cells, and 14 times higher in M2 macrophages compared to M1 macrophages. These results suggest that a pre-existing immunosuppressive microenvironment, associated with resting immune cells and high FCER1A expression, may prevent effective anti-tumor immune responses in ICB non-responders.

Analyses of immune checkpoint and cytokine ligand-receptor interactions revealed coordinated upregulation of several ligand-receptor pairs in the stroma of tumors from ICB responders, including genes encoding PD-L1/PD-L2 and their receptor PD-1, CXCL9/CXCL10 and their receptor CXCR3, and CCL5 and its receptor CCR5 as shown in FIG. 5 and FIG. 7. In contrast, consistent expression changes of checkpoint proteins and cytokines in cancer cells of ICB responders were not found. Among chemokines upregulated in ICB responders, CXCL9, CXCL10, and CXCL13 promote lymphocyte recruitment, while CCL5 is a chemoattractant of both T-cells and myeloid cells. CCL5 also directs the recruitment and differentiation of CCR5+ blood monocytes and neutrophils into immunosuppressive tumor associated macrophages and neutrophils. In addition, CCL5 has direct pro-tumoral effects on cancer cells, promoting cancer proliferation and metastasis. Preclinical and clinical studies suggested that targeting the CCL5-CCR5 axis could enhance anti-tumoral response. As CCL5 and CCR5 are both overexpressed in ICB responders, the results found suggests that targeting CCL5-CCR5 signaling in conjunction with ICB might further potentiate anti-tumoral immune response. Indeed, a recent trial found modest clinical benefit of anti-CCR5 and anti-PD-1 combination on heavily pre-treated microsatellite stable colorectal cancer patients, and more trials are underway to evaluate the safety and efficacy of anti-CCR5 and checkpoint inhibitor combinations. Currently, the majority of companion diagnostics for PD-1/PD-L1 inhibitors measure PD-L1 expression level on either cancer cells alone, or on both cancer cells and infiltrating immune cells, but seldomly on immune cells only. A recent trial found PD-L1 expression on immune cells to be prognostic in head and neck cancer patients treated with ICB. Our study further suggests that PD-L1 expression on immune cells is a more consistent determinant of ICB response across tumor types.

Using the stromal consensus DEGs identified from the meta-analysis, the IOSelect score is developed, which robustly predicted ICB response in 6 independent test cohorts, outperforming existing gene expression signatures for ICB response. Furthermore, we proposed a simple 3-feature model that can attain comparable performance with a single positive marker of immune infiltration (IFNG), a single negative marker of immune suppression (FCER1A), in combination with TMB. While ideal pan-cancer biomarkers of ICB response should be robust across patient cohorts, it was noticed that the Liu et al. melanoma cohort did not share many DEGs with the other cohorts, including other melanoma cohorts. This result highlights significant heterogeneity between ICB cohorts, even among cohorts of the same tumor type. It is unclear to what extent this heterogeneity arise from patient selection, differences in ICB regime, differences in prior treatments, different sample collection protocols, or differences in gene expression assays. This highlights the need for large, consistently selected patient cohorts, with standardized gene expression profiling to further improve biomarker discovery.

As cancer cells are under negative selective pressure from the immune system, several mechanisms of immune escape by cancer cells have been proposed: (i) cancer cells down-regulate antigen presentation by deleting HLA alleles, (ii) cancer cells deplete neoantigens to avoid detection, (iii) oncogenic signaling may up-regulate PD-L1 expression and suppress T-cell recruitment. However, the prevalence of these immune evasion mechanisms are still unclear. A recent meta-analysis found no association between loss of heterozygosity of HLA alleles and ICB response across ICB cohorts. Furthermore, a pan-cancer analysis failed to detect neo-antigen depletion signals in most tumor types. Similarly, we found a lack of conserved cancer-intrinsic mechanisms of immune evasion on the transcriptomic level, which challenge the prevailing dogma that cancer cell-intrinsic expression of checkpoint ligands can modulate immune activity and predict ICB response across cancer types. These insights have profound implications on biomarker assays and in vitro model systems for development of improved ICB drugs. These results suggest that cancer cells adopt diverse mechanisms of immune evasion, with no universal strategy adopted by cancer cells to avoid immune-mediated killing. The growing number of molecularly characterized ICB cohorts will enable further exploration of the importance and prevalence of the different cancer-intrinsic immune evasion strategies in determining ICB response.

### Gene Expression Data Collection and Normalization

Expression data of TCGA colorectal (COAD, READ), gastric (STAD) and endometrial (UCEC) tumors were downloaded from the UCSC Xena repository. Raw genomic and transcriptomic data was obtained from the European Nucleotide Archive, and processed using the bcbio nextgen pipeline. Briefly, RNAseq reads were aligned to the human transcriptome using STAR53, and TPM counts were quantified using SALMON54. Processed transcriptomic data of ICB cohorts were also obtained from the publically available sources. Genes with 0 expression in more than 50% of the samples were removed. All gene expression data were log transformed and upper quantile normalized across all cohorts.

### Tumor Purity Estimation

Purity estimates of TCGA tumors were obtained from a publicly available source noted above, where consensus purity was estimated from mutation allele frequencies, copy number variations and RNA profiles of each tumor. Tumor purity of samples from ICB cohorts were estimated using PUREE, a machine learning method for tumor purity estimation from bulk RNA transcriptomics. For records where genomic data was also available, consensus purity estimates were based on both genomic and transcriptomic data. The purity estimates were computed using 2 RNA-based methods (PUREE and Estimate), and DNA-based methods (Sequenza, PurBayes). Then, quantile-quantile normalization was performed on the purity estimates of individual methods, and the mean normalized purity estimate was selected as the consensus purity.

### MSI Status and ICB Response Classification

MSI status of TCGA tumors were obtained from two previous pan-cancer studies of microsatellite instability. EBV status of the TCGA STAD tumors was obtained from a previous TCGA study on gastrointestinal cancers. Clinical annotations of ICB response based on the RECIST criteria were obtained from the published studies.

### Expression Deconvolution

The bulk tumor expression was modelled as the sum of cancer and stroma expression, weighted by cancer purity. This can be written as,

$${y}_{j} = {c}_{j} p + {s}_{j} ( 1 - p ) + {ε}_{j}$$

where yⱼ is a n×1 vector of bulk tumor expression for gene j in n samples, cⱼ and sⱼ are the average cancer-specific and stroma-specific expression for a gene j, p is n ×1 vector of cancer purity, and εⱼ is the residual. Given bulk expression (yⱼ) and tumor purity (p), non-negative least squares (NNLS) regression can be used to estimate cancer—(cⱼ) and stroma-specific (sⱼ) expression for each gene. cⱼ and sⱼ represent estimates of mean cancer- and stroma-specific expression for a gene j for p_{c}=1 and pₛ=0, respectively. The standard error of these predicted values for gene j is calculated as,

$$S {E}_{j , h} = \sqrt{MSE + ( 1 + \frac{1}{n} + \frac{{( {p}_{h} - \overline{p} )}^{2}}{\sum {( {p}_{i} - \overline{p} )}^{2}} )}$$

where pₕ=p_{c}=1 for cancer-specific expression and pₕ=pₛ=0 for stroma-specific expression; MSE is the mean square error.

### Permutation Statistic to Identify Differentially Expressed Genes

To test differences in cancer (or stroma) expression between two response groups (e.g. c_{Rj}-C_{NRj} for gene j), the above approach is applied to each group separately, and a test statistic is calculated as,

$${Z}_{obs , j}^{2} = {( \frac{{c}_{Rj} - {c}_{NRj}}{\sqrt{S {E}_{Rj}^{2} + S {E}_{NRj}^{2}}} )}^{2}$$

To assess significance under the null hypothesis of no difference in cancer (stroma) expression between the 2 response groups, a null distribution is generated by permutation, where group labels are first randomly shuffled 10,000 times and the above procedure is applied to each permuted dataset, generating a null distribution of Z_perm² statistics. A p-value is calculated as,

$$p = P ( {Z}_{p e r m}^{2} \geq {Z}_{o b s}^{2} )$$

That is, the probability of observing

$${Z}_{o b s}^{2}$$

or larger, given the null hypothesis is true.

### Identification of Consensus DEGs Across Cancer Types

For each gene and each tumor compartment (cancer or stroma), a meta p-value is computed. This computation may involve combining the permutation p-values of all cancer cohorts using Fisher's method. Embodiments used the Bonferroni correction to adjust for multiple testing to maintain the overall α at 0.01. Genes that are differentially expressed in different directions in at least 2 cohorts were removed. Finally, to eliminate DEGs involved in MSI biology but directly associated with ICB response, the final list of consensus DEGs was required to be differentially expressed in at least 1 ICB cohort (nominal p-value<0.1).

### Pathway Enrichment Analysis

To find out if the DEGs identified are enriched in specific biological processes, pathway enrichment analysis was performed using ClueGo6o with default parameters. Briefly, two-sided-hypergeometric statistic was used to compute the enrichment of a given list of DEGs in GO biological pathway terms, and the resulting p-values were corrected for multiple testing using the Bonferroni step down approach. To aid interpretation, significant biological terms with high overlap as measured by the kappa coefficient were merged into functional groups.

To calculate the enrichment of stroma DEGs in immune-related functions, immune-related genes are defined as genes in the “immune system process” term and its child terms in the GO biological processes dataset. Additionally, curated stroma DEGs are manually not labeled as immune genes by GO, and further labeled FCER1A, GZMH, FCRL6, SIRPG, JAKMIP1, and WARS as immune-related genes based on literature search.

### Analysis of Single Cell RNAseq Data

Gene-level normalized counts from Bi et al. were downloaded from the Single Cell Portal. A total of 34,327 cells from 8 renal cell carcinoma tumors were analyzed, of which 17,672 cells from 5 tumors were from ICB-treated patients. Cells with low library size and high mitochondrial contamination were removed. For each annotated cell type, the average expression and proportion of expressing cells was calculated for each DEG, separately for cells from ICB responders and non-responders. To generate the doplot in FIG. 4A, the expression counts of each gene are scaled using z-score normalization, and computed the average scaled expression and the prevalence of expression of each gene across all stromal (non-cancer) cell types.

### Predictive Modelling of ICB Response

The immune score determined using the present response estimation model is defined as the ratio of the mean expression of the top 10 DEGs upregulated in ICB responders (CXCL9, CXCL13, IFNG, KLRD1, GBP1P1, CRTAM, FASLG, GBP5, CD8A, PRF1) to the mean expression of all 8 DEGs down-regulated in ICB responders (FCER1A, PID1, CH25H, NPR3, PREX2, NPR1, FAM134B, SDPR). To build the response estimation model, 461 patients from 3 discovery cohorts are used (Mariathasan et al, Liu et al, Kim et al.) with both RNAseq and TMB as training data.

Whole exome-sequencing FASTQ files of Kim et al. gastric ICB cohort were downloaded from the European Nucleotide Archive, and processed using the bcbio nextgen pipeline. TMB was calculated as the number of nonsynonymous mutations per megabase. For the Mariathasan et al and Liu et al cohorts, TMB was obtained from the original publications. All TMB values were standardized using Z-score transformation (mean=0, standard deviation=1). A logistic regression model is fitted using the immune score generated using the response estimation model and TMB as input features.

Next, experiments are done to build a more compact model with comparable performance. Using the same training data, LASSO is applied for selecting top features (the “top” is a predetermined number, for example, 4 or 10) from the 59 stroma DEGs and TMB. LASSO penalizes the sum of the absolute values of the regression coefficients, shrinking some coefficients to zero. Thus, LASSO results in a simpler and more interpretable model where terms associated with zero coefficients or coefficients with values below a predetermined threshold may be excluded. The regularization parameter \ was chosen by 10-fold cross-validation such that the error of the selected model was within 1 standard deviation from the minimum error. LASSO regression and cross validation were performed using the ‘glmnet’ package in R. To select for the most robust features, 100 samples were bootstrapped with 90% of the data in each bootstrap, and selected features that are found by at least 75% of the bootstraps for the final model. It is found that TMB, IFNG and FCER1A are the most stably selected features. The logistic regression model is then built on all 461 training samples using these three features.

### Benchmarking ICB Prediction Model Against Existing Biomarkers

T-cell GEP scores were calculated from normalized RNAseq data using the weights provided by Ayers et al. 2018. TIDE scores were calculated from normalized RNAseq data using the TIDE web platform. The CXCL-9+TMB model was trained on the training cohort defined herein using logistic regression. The cytolytic score is calculated as the geometric mean of GZMA and PRF1 expression. Vigex score is computed as the mean of z-score scaled expression of 12 genes (CXCL9, CXCL10, CXCL11, IFNG, PRF1, IL7R, GZMA, GZMB, PDCD1, CTLA4, CD274, FOXP3).

### Immune Cell Enrichment Analysis

Immune deconvolution was performed using CibersortX (with the LM22 signature matrix) to estimate the absolute abundance of immune cell subsets for each tumor. Immune cell subsets that are differentially infiltrated between responders and non-responders were identified using the Wilcoxon rank-sum test.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Although this disclosure describes or illustrates particular embodiments as providing particular advantages, particular embodiments may provide none, some, or all of these advantages.

1. A clinical decision support system to predict clinical response of a target tumor to immune checkpoint blockade therapy (ICB), the system comprising one or more processing units to:
(i) generate a response estimation model by:
(a) obtaining a training dataset comprising training transcriptome sequence records of tumor tissue samples of a plurality of known responders and a plurality of known non-responders to ICB;
(b) performing deconvolution of the training dataset to identify differentially expressed genes (DEGs) in the training dataset, wherein the DEGs are treated as features of the training dataset and associated responder, non-responder status is treated as a labels of the records in the training dataset;
(c) performing regression analysis of the features to select a subset of the features as most predictive of the responder or non-responder labels and obtain associated feature weights;
(d) incorporating the subset of features and associated feature weights in the response estimation model;
(ii) receive transcriptome sequence data of a target tumor tissue sample;
(iii) process the received transcriptome sequence data using the generated response estimation model to generate an estimated response metric indicating a likely response of the target tumor to ICB.

2. The system of claim 1, wherein the DEGs are identified based on a p-value for each candidate gene j computed as:
$${p}_{j} = P ( {Z}_{p e r m}^{2} \geq {z}_{obs , j}^{2} )$$
$$wherein$$
$${Z}_{o b s , j}^{2} = {( \frac{{c}_{Rj} - {c}_{NRj}}{\sqrt{S {E}_{Rj}^{2} + S {E}_{NRj}^{2}}} )}^{2} ,$$
$${Z}_{p e r m}^{2}$$
is a null distribution estimated by randomly shuffling the responder and non-responder labels in the training dataset to obtain permutations of the training dataset and computing
$${Z}_{o b s}^{2}$$
for each permuted training dataset to estimate
$${Z}_{perm}^{2} , {c}_{Rj}$$
is the estimated expression level of candidate gene j in responder records of the training dataset with a corresponding standard error SE_{Rj}
C_{NRj} is the estimated expression level of candidate gene j in non-responder records of the training dataset with a corresponding standard error SE_{RNj}, the standard error for candidate gene j being computed as
$$S {E}_{j} = \sqrt{MSE + ( 1 + \frac{1}{n} + \frac{{( {p}_{h} - \overline{p} )}^{2}}{{\sum ( {p}_{t} - \overline{p} ]}^{2}} )}$$
wherein pₕ=p_{c}=1 for cancer-specific gene expression and pₕ=pₛ=0 for stroma-specific gene expression, and MSE is the mean square error, n is the number of records in the training dataset.

3. The system of claim 2, wherein the C_{Rj} and C_{NRj} values are estimated by modelling bulk tumor expression as the sum of cancer and stroma expressions, weighted by cancer purity represented as
$${y}_{j} = {c}_{j} p + {s}_{j} ( 1 - p ) + {ε}_{j}$$
where yⱼ is a n×1 vector of bulk tumor expression for gene j in n samples, cⱼ and sⱼ are the average cancer-specific and stroma-specific expression for a gene j, p is n×1 vector of cancer purity, and εⱼ is a residual.

4. The system of claim 1, wherein the response estimation model also incorporates a tumor mutation burden (TMB) feature; and
the response estimation model processes a TMB metric of the target tumor in generating the estimated response metric.

5. The system of claim 1, wherein the regression analysis is performed using a least absolute shrinkage and selection operator (LASSO).

6. The system of claim 1, wherein the one or more processing units are further configured to:
receive exome sequencing data of the target tumor tissue sample; and
identify clinically actionable genomic mutations associated with ICB by the response estimation model.

7. The system of claim 1, wherein the transcriptome data of a target tumor tissue sample comprises transcriptome data of stromal cells in the tissue sample.

8. A system for generating a response estimation model to predict clinical response of a tumor to immune checkpoint blockade therapy (ICB), the system comprising one or more processing units to:
obtain a training dataset comprising training transcriptome records of tumor tissue samples of a plurality of known responders and a plurality of known non-responders to ICB;
perform deconvolution of the training dataset to identify differentially expressed genes (DEGs) in the training dataset, wherein the DEGs are treated as features of the training dataset and associated responder, non-responder status is treated as a label of the records in the training dataset;
perform regression analysis of the features to select a subset of the features as most predictive of the responder or non-responder labels;
incorporate the subset of features in the response estimation model.

9. A computer-implemented method for predicting clinical response of a tumor to immune checkpoint blockade therapy (ICB), the method comprising:
(i) generating a response estimation model by:
(e) obtaining a training dataset comprising training transcriptome records of tumor tissue samples of a plurality of known responders and a plurality of known non-responders to ICB;
(f) performing deconvolution of the training dataset to identify differentially expressed genes (DEGs) in the training dataset, wherein the DEGs are treated as features of the training dataset and associated responder, non-responder status is treated as a label of the records in the training dataset;
(g) performing regression analysis of the features to select a subset of the features as most predictive of the responder or non-responder labels and obtain associated feature weights;
(h) incorporating the subset of features and associated feature weights in the response estimation model;
(ii) obtaining transcriptome data of a target tumor tissue sample;
(iii) processing the transcriptome data using the generated response estimation model to generate an estimated response metric indicating a likely response of the target tumor to ICB.

10. The method of claim 9, wherein the DEGs are identified based on a p-value for each candidate gene j defined as:
$${p}_{j} = P ( {Z}_{p e r m}^{2} \geq {Z}_{obs , j}^{2} )$$
$$wherein$$
$${Z}_{o b s , j}^{2} = {( \frac{{c}_{R j} - {c}_{N R j}}{\sqrt{S {E}_{Rj}^{2} + {SE}_{NRj}^{2}}} )}^{2} ,$$
$${Z}_{p e r m}^{2}$$
is a null distribution estimated by randomly shuffling the responder and non-responder labels in the training dataset to obtain permutations of the training dataset and computing
$${Z}_{o b s}^{2}$$
for each permuted training dataset to estimate
$${Z}_{perm}^{2} ,$$
C_{Rj} is the estimated expression level of candidate gene j in records responder records of the training dataset with a corresponding standard error SE_{Rj},
C_{NRj} is the estimated expression level of candidate gene j in non-responder records of the training dataset with a corresponding standard error SE_{RNj}, the standard error for candidate gene j being computed as
$$S {E}_{j} = \sqrt{MSE + ( 1 + \frac{1}{n} + \frac{{( {p}_{h} - \overline{p} )}^{2}}{\sum {( {p}_{i} - \overline{p} )}^{2}} )}$$
wherein pₕ=p_{c}=1 for cancer-specific expression and pₕ=pₛ=0 for stroma-specific expression, and MSE is the mean square error, n is the number of records in the training dataset.

11. The method of claim 10, wherein the C_{Rj} and C_{NRj} values are estimated by modelling bulk tumor expression as the sum of cancer and stroma expression, weighted by cancer purity represented as
$${y}_{j} = {c}_{j} p + {s}_{j} ( 1 - p ) + {ε}_{j}$$
where yⱼ is a n×1 vector of bulk tumor expression for gene j in n samples, cⱼ and sⱼ are the average cancer- and stroma-specific expression for a gene j, p is n×1 vector of cancer purity, and εⱼ is the residual.

12. The method of claim 9, wherein the response estimation model also incorporates a tumor mutation burden (TMB) feature; and
the response estimation model processes a TMB metric of the target tumor tissue sample in generating the estimated response metric.

13. The method of claim 9, wherein the regression analysis is performed using a least absolute shrinkage and selection operator (LASSO).

14. The method of claim 9, further comprising:
receiving exome sequencing data of the target tumor tissue sample; and
identifying clinically actionable genomic mutations associated with ICB by the response estimation model.

15. The method of claim 9, wherein the transcriptome data of a target tumor tissue sample comprises transcriptome data of stromal cells in the tissue sample.

16. A computer-implemented method for generating a response estimation model to predict clinical response of a tumor to immune checkpoint blockade therapy (ICB), the method comprising:
obtaining a training dataset comprising training transcriptome records of tumor tissue samples of a plurality of known responders and a plurality of known non-responders to ICB;
performing deconvolution of the training dataset to identify differentially expressed genes (DEGs) in the training dataset, wherein the DEGs are treated as features of the training dataset and associated responder, non-responder status is treated as a label of the records in the training dataset;
performing regression analysis of the features to select a subset of the features as most predictive of the responder or non-responder labels;
incorporating the subset of features in the response estimation model.
